SEQUENCE LISTING (8 sequences)

SEQ ID NO: 1 - protein, 59 aa, artificial sequence
  synthetic blood-brain barrier (BBB) signal peptide
HKKWQFNSPFVPRADEPARKGKVHIPFPLDNITCRVPMAREPTVIHGKREVTLHLHPDH

SEQ ID NO: 2 - protein, 23 aa, artificial sequence
  synthetic blood-brain barrier (BBB) signal peptide
CGGKTFFYGGSRGKRNNFKTEEY

SEQ ID NO: 3 - protein, 23 aa, artificial sequence
  synthetic blood-brain barrier (BBB) signal peptide
CGGKTFFYGGSRGKRNNFKTEEY

SEQ ID NO: 4 - protein, 19 aa, artificial sequence
  synthetic blood-brain barrier (BBB) signal peptide
TFFYGGCRGKRNNFKTEEY

SEQ ID NO: 5 - protein, 19 aa, artificial sequence
  synthetic blood-brain barrier (BBB) signal peptide
TFFYGGSRGKRNNFKTEEY

SEQ ID NO: 6 - protein, 23 aa, artificial sequence
  synthetic blood-brain barrier (BBB) signal peptide
CGGKTFFYGGCRGKRNNFKTEEY

SEQ ID NO: 7 - protein, 23 aa, artificial sequence
  synthetic blood-brain barrier (BBB) signal peptide
CGGKTFFYGGSRGKRNNFKTEEY

SEQ ID NO: 8 - protein, 59 aa, artificial sequence; identical to SEQ ID NO: 1
  synthetic blood-brain barrier (BBB) signal peptide

(19) United States
(12) Patent Application Publication
(10) Pub. No.: US 2014/0213641 A1
(43) Pub. Date: Jul. 31, 2014

(54) POLYMERIC NANOPARTICLES FOR DRUG DELIVERY

(71) Applicants: Salvador Borros Gomez, Barcelona (ES); Primiano Pio Di Mauro, Barcelona (ES)

(72) Inventors: Salvador Borros Gomez, Barcelona (ES); Primiano Pio Di Mauro, Barcelona (ES)

(73) Assignee: INSTITUT QUIMIC DE SARRIA, Barcelona (ES)

(21) Appl. No.: 14/115,991

(22) PCT Filed: May 9, 2012
(86) PCT No.: PCT/IB2012/052320

§ 371 Date: Nov. 6, 2013

(30) Foreign Application Priority Data
May 9, 2011 (GB) 1107719.5
Apr. 3, 2012 (GB) 1205979.6

(51) Int. Cl. A61K 9/51; A61K 31/337

(52) U.S. Cl. CPC A61K 9/5153; A61K 31/337
USPC 514/449; 525/418; 525/54.1; 514/772.1

(57) ABSTRACT

Disclosed are nanoparticles comprising a block copolymer and optionally one or more active agent(s), compositions comprising said nanoparticles and methods of preparing said nanoparticles. The block copolymer comprises blocks (i) a first polymer that is a polyester or polyamide and (ii) a second polymer comprising a hydrocarbon chain containing ester or ether bonds with hydroxyl number ≧10. The active agent(s) may be present within the nanoparticles or on the surfaces of the nanoparticles. The nanoparticles may optionally be associated with a surface-modifying moiety such that they are useful as drug delivery and molecular imaging devices. The surface-modifying moiety may target the nanoparticles to a desired target, cell, tissue or biomarker.

The invention is in the field of nanoparticles comprising a block copolymer. The invention also pertains to nanoparticles that may have incorporated an active agent and optionally be associated with a surface-modifying moiety such that they are useful as drug delivery and molecular imaging devices. The invention also pertains to methods for preparing such nanoparticles and methods for modification of their surfaces.

Biodegradable nanoparticles have been used as sustained release vehicles for administering active agents such as natural or synthetic organic or inorganic entities, proteins, peptide and nucleic acids. The active agent is dissolved in, entrapped in, encapsulated in or attached to a nanoparticle matrix. Biodegradable nanoparticles, particularly those coated with hydrophilic polymer such as poly(ethylene glycol) (PEG), are useful as drug delivery devices as they circulate for a prolonged period and may target a particular site for delivery (Mohanraj & Chen Trop. J. Pharm. Res. 5, 561-573 (2006)).

The major goals in designing nanoparticles as a delivery system are to control particle size, surface properties and release of pharmacologically active agents in order to achieve site-specific action of the drug at the therapeutically optimal rate and dosage regimen. Nanoparticles can be prepared from a variety of materials such as proteins, polysaccharides and synthetic polymers. The selection of matrix materials is dependent on many factors including the size of nanoparticles required, the inherent properties of the encapsulated drug (for example, aqueous solubility and stability), the surface characteristics (such as charge and permeability), the degree of biodegradability, biocompatibility and toxicity, the drug release profile desired, and the antigenicity of the final product.

Although liposomes have been used as potential carriers with advantages including protecting drugs from degradation, targeting to site of action and reduced toxicity or side effects, their applications may be limited by problems such as low encapsulation efficiency, rapid leakage of water-soluble drug in the presence of blood components and poor storage stability. Nanoparticles offer some specific advantages over liposomes. For instance, they are more stable during storage, help to increase the stability of drugs and proteins and possess useful controlled release properties.

The advantages of using nanoparticles as a drug delivery system are manifold. The particle size and surface characteristics of nanoparticles can be easily manipulated to achieve both passive and active drug targeting after systemic passage. They control and sustain release of the drug during the transportation and at the site of localization, altering organ distribution of the drug and subsequent clearance of the drug so as to achieve an increase in drug therapeutic efficacy and a reduction in side effects by minimising interaction with other organs. Controlled release and particle degradation characteristics can be readily modulated by the choice of matrix constituents. Drug loading is relatively high and drugs can be incorporated into the systems without any chemical reaction; this is an important factor for preserving the drug activity. Site-specific targeting can be achieved by attaching targeting ligands to the surface of particles or use of magnetic guidance. The size, surface charge and surface decoration of the nanoparticles can be modulated. The system can be used for various routes of administration including oral, nasal, parenteral, pulmonary, vaginal and intraocular.

A continuing need exists for the development of new nanoparticles for drug delivery that can be tuned for precise release profiles and are able to encapsulate a wider range of active agents, including polar active agents, at higher weight percentages of the nanoparticles. New methods for modifying the surface of these nanoparticles are also desirable. Nanoparticles that may function as vectors for delivery of active agents to the brain are also desirable.

The invention provides a nanoparticle comprising a block copolymer, and optionally one or more active agent(s), wherein:
(i) the block copolymer comprises blocks A and D;
(ii) block A consists of a first polymer comprising monomer units B and C, wherein B is an aliphatic dicarboxylic acid wherein the total number of carbon atoms is ≦30 and C is a dihydroxy or diamino monomer; and
(iii) block D consists of a second polymer comprising a hydrocarbon chain containing ester or ether bonds with hydroxyl number ≧10.

The present invention further provides a composition, particularly a pharmaceutical composition, comprising a nanoparticle wherein said nanoparticle comprises a block copolymer and optionally one or more active agent(s) and wherein:
(i) the block copolymer comprises blocks A and D;
(ii) block A consists of a first polymer comprising monomer units B and C, wherein B is an aliphatic dicarboxylic acid wherein the total number of carbon atoms is ≦30 and C is a dihydroxy or diamino monomer;
(iii) block D consists of a second polymer comprising a hydrocarbon chain containing ester or ether bonds with hydroxyl number ≧10; and
(iv) the composition optionally further comprises a vehicle.

The present invention further provides a composition comprising a mixture of (i) nanoparticles that comprise a block copolymer described herein and (ii) nanoparticles that comprise a different block copolymer described herein,

The nanoparticles of the present invention are capable of loading with active agents of widely varying polarity. The active agent, if present, may be incorporated into the nanoparticles, for instance by adsorption, absorption or entrapment, and released from the nanoparticles for instance by desorption, diffusion, polymer erosion, enzyme-mediated release, nanoparticle disintegration for accelerated release, or some combination of these mechanisms.

The active agent(s) may be present within the nanoparticles or on the surfaces of the nanoparticles. The interaction between the active agent(s) and the nanoparticle is typically non-covalent, for example, hydrogen bonding, electrostatic interactions or physical encapsulation. However, in an alternative embodiment, the active agent(s) and the nanoparticle are linked by a covalent bond or linker.

A further advantage of the nanoparticles of the present invention is the prevention of burst release of an incorporated active agent. Early burst release of an active agent from a controlled delivery system following administration can lead to toxic levels of the active agent or prevent the active agent reaching its targeted site of interest. The biodegradability of the polymer, and therefore the release profile of the nanoparticles, may be tuned by modifying the number of monomers in blocks A and D; the ratio of molecular weights of the blocks; the total molecular weight of the polymer; or the hydrophilicity of the polymer. For example, the length of block A may be varied to obtain a longer or shorter release profile. Excipients such as polysorbates, esters of sorbitan with fatty acids, sugars and lipases may also be encapsulated within the nanoparticle.

The nanoparticles may further comprise a disintegrant, superdisintegrant or wetting agent to aid release of the active agent. Alternatively, the nanoparticles may include water-soluble molecules that dissolve to form pores or channels in the nanoparticles through which the active agent may be released.

A further advantage of the nanoparticles of the present invention is that they allow pH-independent release such that release of the active agent is not affected by the different pH environments in the body, for example in the gastrointestinal tract. pH-independent release is defined herein as a variation of less than 10% in the rate of active agent diffusing from the nanoparticles in environments with pH from 1 to 9.

The nanoparticles are biocompatible and sufficiently resistant to their environment of use that a sufficient amount of the nanoparticles remain substantially intact after entry into the mammalian body so as to be able to reach the desired target and achieve the desired physiological effect. The block copolymers and their constituent blocks described herein are biocompatible and preferably biodegradable.

As used herein, the term ‘biocompatible’ describes as substance which may be inserted or injected into a living subject without causing an adverse response. For example, it does not cause inflammation or acute rejection by the immune system that cannot be adequately controlled. It will be recognized that “biocompatible” is a relative term, and some degree of immune response is to be expected even for substances that are highly compatible with living tissue. An in vitro test to assess the biocompatibility of a substance is to expose it to cells; biocompatible substances will typically not result in significant cell death (for example, >20%) at moderate concentrations (for example, 50 μg/10⁶ cells).

As used herein, the term ‘biodegradable’ describes a polymer which degrades in a physiological environment to form monomers and/or other non-polymeric moieties that can be reused by cells or disposed of without significant toxic effect. Degradation may be biological, for example, by enzymatic activity or cellular machinery, or may be chemical. Degradation of a polymer may occur at varying rates, with a half-life in the order of days, weeks, months, or years, depending on the polymer or copolymer used.

The nanoparticles are also haemocompatible. Haemocompatibility may be determined according to ISO 10993-4. Compositions comprising nanoparticles of the present invention may be readily prepared to be endotoxin-free (preferably <2 EU/ml by the Limulus Amebocyte Lysate (LAL) test). Further, empty nanoparticles show low cytotoxicity (preferably IC₅₀ >1 μM, more preferably >10 μM, more preferably >100 μM, more preferably >1 mM towards cancer and non-cancer cells).

As used herein, the term ‘nanoparticles’ refers to a solid particle with a diameter of from about 1 to about 1000 nm. The mean diameter of the nanoparticles of the present invention may be determined by methods known in the art, preferably by dynamic light scattering. In particular, the invention relates to nanoparticles that are solid particles with a diameter of from about 1 to about 1000 nm when analysed by dynamic light scattering at a scattering angle of 90° and at a temperature of 25° C., using a sample appropriately diluted with filtered water and a suitable instrument such as the Zetasizer™ instruments from Malvern Instruments (UK) according to the standard test method ISO 22412:2008 (cumulants method A.1.3.2). Where a particle is said to have a diameter of x nm, there will generally be a distribution of particles about this mean, but at least 50% by number (e.g. >60%, >70%, >80%, >90%, or more) of the particles will have a diameter within the range x±20%.

Preferably, the diameter of the nanoparticle is from about 10 to about 1000 nm, more preferably from about 5 to about 500 nm, more preferably from about 50 to about 400 nm, more preferably from about 50 to about 150 nm. Alternatively, the diameter of the nanoparticle is from about 1 to about 100 nm. In one embodiment, the nanoparticles exhibit a degree of agglomeration of less than 10%, preferably less than 5%, preferably less than 1%, and preferably the nanoparticles are substantially non-agglomerated, as determined by transmission electron microscopy.

The nanoparticles of the present invention may be provided in an acceptable pharmaceutical composition for mammalian and particularly human use. They are typically provided in a vehicle. The vehicle is typically a liquid and forms a continuous phase in the composition. Thus, the preferred composition of the present invention is a dispersion of nanoparticles in a liquid vehicle that comprises the continuous phase of the composition. In particular, the vehicle is one which allows transport of said nanoparticles to a target within the mammalian body after administration. The vehicle may be any pharmaceutically acceptable diluent or excipient, as known in the art. The vehicle is typically pharmacologically inactive. Preferably, the vehicle is a polar liquid. Particularly preferred vehicles include water and physiologically acceptable aqueous solutions containing salts and/or buffers, for example, saline or phosphate-buffered saline. Optionally, the vehicle is a biological fluid. A liquid vehicle may be removed by, for example, lyophilization, evaporation or centrifugation for storage or to provide a powder for pulmonary or nasal administration, a powder for suspension for infusion, or tablets or capsules for oral administration.

The choice of vehicle will be influenced by factors such as the intended mode of administration of the composition. For example, a solid vehicle may be used to provide a powder for pulmonary or nasal administration, a powder for suspension for infusion, or tablets or capsules for oral administration; and a liquid vehicle may be used to provide a suspension for intravenous infusion or a solution for nasal administration.

Preferably, the nanoparticles constitute from about 1% to about 90% by weight of the composition. More preferably, the nanoparticles constitute about 5% to about 50% by weight of the composition, more preferably, about 10% to about 30%.

The nanoparticles of the present invention may also find use in other fields than medicine and drug delivery, for example, agriculture, electronics, paints and adhesives.

The block copolymer comprises at least one block A and at least one block D. Where there are a plurality of block A and/or block D recurring units, each block A and/or each block D may be identical throughout the block copolymer or the block copolymer may comprise different types of block A and/or different types of block D, within the definitions herein. Variations in the identity of blocks A and D include the identity of the monomers (i.e. the chemical composition) and the molecular weight of each block. Similarly, each monomer, B and C, in any block A may be identical throughout the block or the block may comprise independently selected monomers falling within the definitions herein. The block copolymer may be a random block copolymer. In a preferred embodiment, each block A in the copolymer has the same chemical composition, and/or each block D has the same chemical composition. Preferably, each block A has the same molecular weight or molecular weight distribution, and/or each block D has the same molecular weight or molecular weight distribution.

Preferably, the block copolymer is a rigid-flexible block copolymer, wherein A is a rigid block and D a flexible block. The block copolymer may be terminated only by blocks A, or only by blocks D, or by a mixture of blocks A and D. Preferably, the block copolymer is terminated at each end by a block D. Preferably, A is a hydrophobic block and D is a hydrophilic block.

Preferably, A has the formula —[(B—C)ₙ—B]— or —[(C—B)ₙ—C]— wherein n is a numerical index of at least 1, selected independently for each block A. Where A has the formula —[(C—B)ₙ—C]—, a linking group may be employed to join block A to block D. The linking group may be a dicarboxylic acid. Preferably, A has the formula —[(B—C)ₙ—B]—. Preferably, n is at least 5, more preferably it is from 5 to 20, more preferably it is from 5 to 15.

Preferably, B contains from 2 to 20 carbon atoms, more preferably from 2 to 15 carbon atoms, more preferably from 4 to 10 carbon atoms. Alternatively, B contains from 5 to 20 carbon atoms, more preferably from 5 to 10 carbon atoms. Preferably, B is a straight-chain saturated dicarboxylic acid. B may contain ≧2 functional groups. Preferably, B is selected from the group comprising succinic acid, glutaric acid, adipic acid, pimelic acid, suberic acid, azelaic acid and sebacic acid, preferably from glutaric acid, adipic acid, pimelic acid, suberic acid, azelaic acid and sebacic acid, and more preferably from glutaric acid and adipic acid. In one embodiment, B is a straight-chain dicarboxylic acid containing one or more carbon-carbon double bond(s), such as maleic acid, fumaric acid or glutaconic acid.

Preferably, C is an aliphatic diamine or diol containing ≦30 carbon atoms, preferably containing from 4 to 10 carbon atoms. Preferably, C is a straight-chain aliphatic diol, preferably containing from 2 to 15, more preferably containing from 4 to 10 carbon atoms, more preferably being 1,8-octanediol. Alternatively, C is a straight-chain aliphatic diamine, preferably containing from 2 to 15, more preferably from 4 to 10 carbon atoms.

Preferably, the block D is chosen from the group of polyalkylene glycols (particularly polyethylene glycol), polyamidoamines, polyamines, polyols and combinations thereof. Preferably, the block D is selected from a polyalkylene glycol, preferably a polyethylene glycol (PEG).

The molecular weight of the polymer D is preferably 150-20,000 kDa, more preferably 1500-10,000 kDa, more preferably 2000-3000 kDa. The molecular weight of the polymer D is preferably 150-20,000 Da, more preferably 1500-10,000 Da, more preferably 2000-3500 Da. The molecular weight of polymer D may be 150 Da, 200 Da, 300 Da, 400 Da, 600 Da, 1000 Da, 1450 Da, 1500 Da, 3350 Da, 4000 Da, 6000 Da or 8000 Da.

The molecular weight of the blocks may be chosen to modulate the nanoparticle characteristics such as the active agent affinity and the resulting encapsulation efficiency, active agent release kinetics, water uptake and nanoparticle degradation. For example, the relative average lengths of blocks A and D may be altered in order to modulate the hydrophilicity/lipophilicity ratio in the block copolymer and thus the release profile of the active agent. In an embodiment, n is from 5 to 20 or from 5 to 15 and block D is of molecular weight 2500-5000 Da.

The block copolymer employed in the present invention can be synthesized by conventional techniques known in the art. A preferred method comprises the following steps: (i) reacting monomer units B with monomer units C, preferably in proportions such that B is located at the termini of the resulting blocks A; (ii) reacting block A with block D to produce the block copolymer, preferably in proportions such that D is located at the termini of the resulting block copolymer. The reactions may be carried out, for example, by use of microwave irradiation (that is, with a wavelength of from 1 mm to 1 m) as an energy source.

The block copolymer employed in the present invention can be used to produce nanoparticles. The block copolymer has the advantage that it is suitable for use in a wide variety of methods for production of nanoparticles. The nanoparticles of the present invention may be produced by methods known in the art, which may be divided into two main categories: (i) formation including a polymerization reaction; and (ii) formation by dispersion of a preformed copolymer.

Formation of nanoparticles including a polymerization reaction can be further classified into emulsion and interfacial polymerization. Emulsion polymerization may be organic or aqueous, depending on the continuous phase.

Formation of nanoparticles by dispersion of a preformed copolymer can include the following techniques: emulsification/solvent evaporation, solvent displacement and interfacial deposition, emulsification/solvent diffusion, and precipitation by increasing salt concentration. In these techniques, the block copolymer is first produced then processed further to form the nanoparticles.

The methods may utilize interfacial condensation, supercritical fluid processing techniques, ionic gelation or coacervation for the production of the nanoparticles.

Where the nanoparticles of the present invention comprise an active agent, the active agent may be present during the production of the nanoparticles, typically wherein the active agent(s) are present in a liquid medium used for the production of the nanoparticles. Alternatively, or additionally, the active agent(s) may be incorporated by absorption into the nanoparticles after their production.

Preferably, the nanoparticles are formed by dispersion of a preformed copolymer using the technique of solvent displacement and interfacial deposition. The solvent displacement method (Fessi et al. Int. J. Pharmaceutics 55, R1-R4 (1989)) has been used for the formation of nanoparticles. Bilati et al. (Eur. J. Pharm. Sci. 24, 67-75 (2004)) describes the approaches that have been taken to achieve encapsulation of hydrophilic drugs by this method.

The solvent displacement method does not require high stirring rates, sonication or very high temperatures. For example, it may be carried out at 25° C. and at stirring rates of 50-150 rpm, more preferably about 100 rpm. It is characterized by the absence of an oily-aqueous interface, reducing the likelihood of damage to the active agent(s). The procedure may be carried out without use of surfactants, and without the use of organic solvents that may be toxic and therefore incompatible with pharmaceutical and veterinary applications if residues in excess of acceptable limits remain in the nanoparticles.

The solvent displacement method uses two solvents that are miscible and constitute a diffusing medium and a dispersing medium. Preferably, the copolymer and, if present, the active agent(s) are soluble in the diffusing medium (typically referred to as “the solvent”) but neither is soluble in the dispersing medium (typically referred to as “the non-solvent”). The copolymer and optionally the active agent(s) are dissolved in the diffusing medium and the resulting solution is added to the dispersing medium. Optionally, the dispersing medium includes a surfactant. As soon as the diffusing medium has diffused into the dispersing medium, nanoprecipitation occurs by a rapid desolvation of the copolymer, forming nanoparticles in which the active agent is sited within the copolymer. The diffusing medium is preferably added directly to the dispersing medium, for example via syringe, in order to avoid introduction of an air-liquid interface into the process. Various methods are available for separating the nanoparticles from the dispersing and diffusing media, for example, lyophilization, tangential filtration, centrifuge and ultra-centrifuge, or a combination of these methods. In some cases, for example when the nanoparticles are large, centrifugation is preferred. In some cases, for example in the preparation of large batches, the nanoparticle composition may be concentrated by tangential filtration then lyophilized. Preferably, the dispersing and diffusing media are removed by centrifugation or rotary evaporation. The particles are optionally resuspended in a solvent to remove adhered active agent from the surface of the nanoparticles. This solvent may be removed by a further centrifugation step. The nanoparticles may finally be resuspended in a suitable polar liquid.

Thus, a preferred method (a solvent displacement method) for preparing the nanoparticles of the present invention comprises:
i) dissolving the block copolymer and if present the active agent(s) in a diffusing medium to form a first solution;
ii) mixing said first solution with a dispersing medium to form precipitated nanoparticles comprising said block copolymer and if present said active agent(s), and a liquid phase comprising the diffusing and dispersing media; and
iii) separation of the nanoparticles from the liquid phase,
   wherein the diffusing medium comprises a solvent in which the block copolymer and if present the active agent(s) is soluble, wherein the dispersing medium comprises a solvent in which the block copolymer and if present the active agent(s) is not soluble, and wherein the diffusing medium and the dispersing medium are miscible.

The nanoparticles of the present invention may be synthesized in the presence or absence of an active agent for encapsulation. The block copolymer is sufficiently hydrophobic to be insoluble in water and is capable of appropriate hydrogen bonding for nanoparticle formation both with an active agent and with itself.

A preferred method for preparing the composition of the present invention comprises said method for preparing the nanoparticles, and further comprises the step of:
iv) re-suspending the nanoparticles in a vehicle.

The invention further provides a method for preparing the nanoparticles and compositions defined herein, wherein the method comprises use of at least one liquid medium comprising the active agent(s), preferably wherein the active agent(s) are dissolved therein.

The solvent displacement method described herein enables modulation of the properties of the nanoparticles by selection of the parameters of the process and the properties of the components used therein. In particular, the nanoparticle size, polydispersivity, zeta-potential, active agent encapsulation efficiency, active agent entrapment, release profile of the active agent(s) and degradation profile of the nanoparticle may be controlled. The zeta-potential is preferably from −45 mV to +20 mV, more preferably from about −40 mV to about −20 mV. Alternatively the zeta-potential may be between −20 mV and +20 mV.

Herein, the active agent encapsulation efficiency refers to the active agent incorporated into the nanoparticles as a weight percentage of the total active agent used in the method of preparation of the active agent-containing nanoparticles. It is typically up to and including 95%, more typically from 70% to 95%.

Herein, active agent entrapment refers to the weight percentage of the active agent in the active agent-loaded nanoparticles. Active agent entrapment is preferably at least 2 wt %, more preferably at least 5 wt %, more preferably at least 10 wt % and typically in the range of from 2 wt % to 20 wt %, more preferably from 5 wt % to 20 wt %, more preferably from 10 wt % to 20 wt %.

It is an advantage of the block copolymer employed in producing the nanoparticles of the present invention that it allows high active agent entrapment. Active agent entrapment is greater than that previously demonstrated with other nanoparticles. For example, where nanoparticles of the present invention are produced by a solvent displacement method, active agent entrapment is from 1 to 10 wt % or from 2 to 5 wt %, whereas production of nanoparticles known in the art by solvent displacement allows entrapment of ˜1 wt %. Preferably active agent entrapment is >4 wt %. Where nanoparticles of the present invention are produced by a double emulsion method, active agent entrapment is typically at least 5 wt % and preferably at least 10 wt %. By contrast, production of nanoparticles from other materials by double emulsion methods provides active agent entrapment of only about 3-4 wt %.

Nanoparticles of the present invention may be formed with high active agent content (e.g. >5%) and high encapsulation efficiency (e.g. 70-95%)

Variation of the non-solvent, solvent:non-solvent ratio, polymer concentration, percentage of dissolved drug and the method of separating the nanoparticles from the medium may be used to modulate these properties.

The solvent is suitably selected from liquids in which the polymer and, if present, the active agent are soluble. It is preferably a polar, aprotic solvent. Preferred solvents include acetone, methylethyl ketone, methyl propyl ketone, acetonitrile, dimethylformamide, dimethylsulfoxide, 2-pyrrolidone and N,N-dimethylacetamide or mixtures thereof. The non-solvent is suitably selected from liquids in which the polymer and, if present, the active agent(s) are insoluble. Preferred non-solvents include water, methanol and ethanol, or mixtures thereof. Any substance deemed acceptable in the European Medicines Agency Guidelines Reference Number EMA/CHMP/ICH/82260/2006 may be used as a solvent or non-solvent. A buffer may be used to obtain a pH at which the active agent is not soluble. The identity of the non-solvent influences the size of nanoparticles obtained. The solvent and non-solvent are preferably present in a volume ratio of from 1:1 to 1:50 solvent:non-solvent, more preferably from 1:2 to 1:20, more preferably 1:10.

The concentration of the block copolymer in the diffusing medium is not limited. However, preferably it is from 1 to 1000 mg/ml, more preferably 5 to 100 mg/ml, more preferably from 10 to 50 mg/ml, more preferably 20 mg/ml. If the polymer concentration is too high, this may prevent the formation of nanoparticles.

The concentration of the active agent, or of each active agent where more than one is present, in the diffusing or dispersing medium is preferably from 1 to 500 mg/ml, more preferably 5 to 100 mg/ml, more preferably from 10 to 50 mg/ml, more preferably 20 mg/ml. A higher concentration of active agent results in a higher active agent encapsulation efficiency and higher active agent entrapment.

A further method for preparing the nanoparticles comprises:
i) dissolving the block copolymer in a water-immiscible solvent;
ii) dissolving the active agent, if present, in a water-miscible solvent;
iii) forming a water-in-oil emulsion; and
iv) evaporation of the first solvent to form nanoparticles;
   wherein the water-immiscible solvent and the water-miscible solvent are immiscible.

A further method for preparing the nanoparticles (a double emulsion method) comprises:
i) dissolving the block copolymer in a water-immiscible solvent;
ii) dissolving the active agent, if present, in a water-miscible solvent;
iii) forming a water-in-oil emulsion;
iv) dispersing said water-in-oil emulsion in a water-miscible solvent containing a polymeric surfactant;
v) forming a water-in-oil-in-water emulsion; and
vi) filtering the water-in-oil-in-water emulsion to obtain nanoparticles;
   wherein the water-immiscible solvent and the water-miscible solvent(s) are immiscible.

A further method for preparing the nanoparticles (modified double emulsion method) includes the following steps:
i) dissolving the block copolymer in a water-immiscible solvent;
ii) dissolving the active agent, if present, in a water-miscible solvent;
iii) forming a oil-in-water emulsion;
iv) dispersing said oil-in-water emulsion in a water-immiscible solvent containing a polymeric surfactant;
v) forming a oil-in-water-in-oil emulsion; and
vi) filtering the oil-in-water-in-oil emulsion to obtain nanoparticles;
   wherein the water-immiscible solvent and the water-miscible solvent(s) are immiscible.

Herein, “active agent” means a bioactive or therapeutic moiety that causes a biological effect when administered to an animal. Any active agent for which delivery to the mammalian body is desirable is contemplated for association with, or incorporation in, the nanoparticle of the present invention. The nanoparticles of the present invention can comprise one or more active agents, and in one embodiment comprise only one active agent. The active agent may be lipophilic or hydrophilic and may be a natural or synthetic organic or inorganic entity, protein (including antibodies, antibody fragments and interferons), peptide, nucleic acid, lipid or polysaccharide. Preferably the at least one active agent is selected from the group comprising paclitaxel and docetaxel. Preferably the at least one active agent comprises paclitaxel.

When the nanoparticles of the present invention have incorporated an active agent, said nanoparticles display favourable characteristics, for example, similar or higher efficacy, compared with the active agent alone. Where the active agent is a cytotoxic agent, for example paclitaxel, the nanoparticles show similar or higher antitumor activity but similar or decreased toxicity to healthy cells.

When the nanoparticles are produced by the solvent displacement method, the identity of the active agent is limited only by its solubility in the diffusing medium. If the solubility is too high it will not be incorporated in the nanoparticle. However, it is an advantage of the block copolymer employed in producing the nanoparticles of the present invention that it allows an increased range of drugs that may be encapsulated. Thus, the active agent preferably has a logP value of −1.0 to +5.6. For example, hydrophobic active agents with logP values of from +3.0 to +5.6 may be used in the present invention. Hydrophilic active agents with logP values of from −1.0 to +3.0 may also be used.

The nanoparticles may comprise a combination of two or more active agents. For example, more than one active agent may be incorporated within the nanoparticle, and/or more than one active agent may be adhered to the surface of the nanoparticle. A mixture of nanoparticles comprising a first active agent (or first mixture of active agents) and nanoparticles comprising a second active agent (or second mixture of active agents) is within the scope of the invention.

The nanoparticles may comprise a first active agent fraction and a second active agent fraction. The first active agent fraction may be incorporated within the nanoparticle and the second active agent fraction may be adsorbed on the surface of the nanoparticle. An active agent or active agent fraction may have a specific release profile, for example, it may be immediate release, non-immediate release or delayed release. Preferably the rate of release is approximately zero order (i.e. independent of time) over at least 80% of the release period, more preferably over at least 90% of the release period.

The release profile of the active agent from the nanoparticles may be determined by a dialysis method. For example, in an aqueous medium containing 1 M sodium salicylate, 1 ml of active agent-loaded nanoparticle solution (containing 0.1 mg active agent) is introduced into a dialysis bag (MWCO 14000 Da, containing 1 M sodium salicylate by dialysis method) and the end-sealed dialysis bag is submerged fully into 50 ml of 1 M sodium salicylate solution at 37° C. with stirring at 100 rpm for 96 h. At appropriate time intervals, 0.2 ml aliquots were withdrawn and replaced with an equal volume of fresh medium. The concentration of active agent in samples was determined by HPLC with correction for the volume replacement.

The term “immediate release” indicates that, for example, after 12 hours, at least 50% of the active agent or active agent fraction has been released, preferably at least 70%, more preferably at least 90%. Alternatively, it may indicate that, after 24 hours, at least 50% of the active agent or active agent fraction has been released, preferably at least 70%, more preferably at least 90%.

The term “non-immediate release” indicates that, for example, after 12 hours, less than 50% of the active agent or active agent fraction has been released, preferably less than 70%, more preferably less than 90%. Alternatively, it may indicate that, after 24 hours, less than 50% of the active agent or active agent fraction has been released, preferably less than 70%, more preferably less than 90%.

The term “delayed release” indicates that, for example, after 24 hours, less than 50% of the active agent or active agent fraction has been released, preferably less than 40%, more preferably less than 30%. Alternatively, it may indicate that, after 48 hours, less than 50% of the active agent or active agent fraction has been released, preferably less than 40%, more preferably less than 30%, even more preferably less than 20%.

The first active agent fraction may have a different release profile to that of the second active agent fraction. For example, the first active agent fraction may be a delayed release fraction and the second active agent fraction may be an immediate release fraction, or vice versa. The active agent(s) comprised in the first active agent fraction may be the same or different from the active agent(s) comprised in the second active agent fraction.

For example, the nanoparticles may comprise a first active agent fraction incorporated within the nanoparticle and the second active agent fraction adsorbed on the surface of the nanoparticle, wherein the first active agent fraction and the second active agent fraction comprise the same active agent. In this case, the first active agent fraction may be a delayed release fraction and the second active agent fraction may be an immediate release fraction. In this case, preferably less than 30% of the delayed release fraction is released after 48 hours.

Where the first active agent fraction and the second active agent fraction comprise the same active agent(s), the ratio (wt:wt) of the first active agent fraction to the second active agent fraction may be from 20:1 to 1:1, from 10:1 to 1:1, from 2:1 to 1:1, from 1:1 to 2:1, from 1:1 to 10:1 or from 1:1 to 20:1.

Alternatively, a mixture of (i) nanoparticles with specific active agent release profile and (ii) nanoparticles with different active agent release profile is within the scope of the invention. The nanoparticles with different release profiles may comprise different or the same active agent(s).

The invention further provides a method for preparing nanoparticles comprising one or more active agent(s) as defined herein, said method comprising the following steps:
i) producing nanoparticles;
ii) incubating said nanoparticles with a concentrated solution of the active agent(s); and
iii) separating the nanoparticles comprising said active agent(s) from the liquid phase.

The invention further provides a method for preparing the composition of the present invention wherein the nanoparticles comprise one or more active agent(s), said method comprising the following steps:
i) producing nanoparticles;
ii) incubating said nanoparticles with a concentrated solution of the active agent(s);
iii) separating the nanoparticles comprising said active agent(s) from the liquid phase; and
iv) re-suspending the nanoparticles in a vehicle,

The nanoparticles of the present invention may advantageously comprise one or more surface-modifying agent(s) for the purpose of modulating the pharmacological properties thereof. The surface-modifying agents contemplated for use in the present invention include diagnostic agents, targeting agents, imaging agents and therapeutic agents. Positively charged surface-modifying agents may be used. The surface-modifying agents may be polypeptides, polynucleotides, polysaccharides, fatty acids, lipids, and natural and synthetic small molecules. A mixture of nanoparticles comprising a different surface-modifying agent(s) is within the scope of the invention.

A mixture of (i) nanoparticles comprising a surface-modifying agent, for example a surface-modifying agent that is a targeting agent for the blood-brain barrier, and (ii) nanoparticles comprising no surface-modifying agent is within the scope of the invention. Such a mixture could be used to treat both a secondary tumour in the brain and a primary tumour in another part of the body such as lung or breast.

Targeting agents direct the nanoparticle to a desired target, cell, tissue or biomarker and may recognize disease-related biomarkers on the surface of cells. They may include signal peptides, antibodies and aptamers. Targeting agents will vary depending on the target and suitable targeting agents will be readily available to the skilled person. Preferred targeting agents include thiolated polymers (e.g. to improve mucosal adhesion), blood-brain barrier (BBB) signal peptides and cell adhesion peptides, including but not limited to RGD, RGDC, RGDV and RGDS peptides (e.g. for targeting to integrin receptors). The surface-modifying agent may be a peptide, preferably SEQ ID #1.

Nanoparticles of the present invention are able to cross the BBB. Where the nanoparticle of the present invention comprises a surface-modifying agent (i.e. a targeting agent) that is a BBB signal peptide, a signal nanoparticle may act as a nanoshuttle, delivering multiple active agent moeities across the BBB. Preferred BBB signal peptides include peptides comprising SEQ ID #1, 2, 3, 4, 5, 6, 7 and 8 shown one-letter code in Table 1 (5-TAMRA representing 5-carboxytetramethylrhodamine; BIO representing biotin, CARB representing a saccharide).

TABLE 1

| SEQ ID # | Peptide sequence |
|---|---|
| 1 | (5-TAMRA-)HKKWQFNSPFVPRADEPARKGKVHIPFPLDNI- |
| | TCRVPMAREPTVIHGKREVTLHLHPDH |
| 2 | 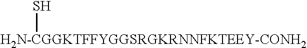 |
| 3 | 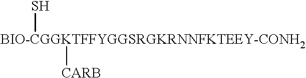 |
| 4 | TFFYGGCRGKRNNFKTEEY |
| 5 | TFFYGGSRGKRNNFKTEEY |
| 6 | CGGKTFFYGGCRGKRNNFKTEEY |
| 7 | CGGKTFFYGGSRGKRNNFKTEEY |
| 8 | HKKWQFNSPFVPRADEPARKGKVHIPFPLDNITCRVPMAREPTVIHGKREVTLHL |
| | HPDH |

Diagnostic and imaging agents include contrast agents, magnetic materials, agents sensitive to light, radiolabels, and fluorescent compounds, such as carboxyfluorescein. Such agents may be used for biodistribution studies in vitro and in vivo. Delivery of nanoparticles of the present invention to the brain has been demonstrated by such studies. For example, paclitaxel-loaded nanoparticles comprising surface-modifying agents have been detected in the brain in biodistribution studies in vivo. Moreover, fluorescently labelled nanoparticles can be used in a cellular study simulating the blood-brain barrier.

A further example of a surface-modifying agent is biotin.

The surface-modifying agent may be introduced into or onto the nanoparticle via contact with a preformed nanoparticle, or with the block copolymer or one of its constituent polymers or monomers prior to nanoparticle formation. Association of the surface-modifying agent with the nanoparticle or block copolymer may be by covalent attachment, electrostatic interaction or specific or non-specific adsorption.

Accordingly, nanoparticles of the present invention are particularly versatile in the range of surface-modifying agents that may be coupled to them.

In a preferred embodiment of the present invention, the surface-modifying agent is introduced into or onto the nanoparticle or block copolymer via a coupling agent. Thus, according to a further aspect of the invention, the nanoparticles defined herein have a coupling agent introduced into or onto the nanoparticles. A coupling agent allows association of a surface-modifying agent of interest with the nanoparticle. Typically, all or part of the coupling agent is retained when the surface-modifying agent is associated with the nanoparticle.

The surface-modifying agent may be coupled to the block copolymer before or after nanoparticle formation. Where a surface-modifying agent is a peptide attached to the block copolymer before nanoparticle formation, it is typically situated on the surface of a nanoparticle formed by the solvent displacement method. Where a surface-modifying agent is hydrophobic and attached to the block copolymer before nanoparticle formation, it is typically situated within a nanoparticle formed by the solvent displacement method. A surface-modifying agent such as a radiolabel may be usefully situated within a nanoparticle.

Preferably, the nanoparticle is formed from the block copolymer comprising a modified polymer to which a coupling agent containing a sulfhydryl-reactive group is attached. Alternatively, the nanoparticle is formed from the block copolymer comprising a modified polymer to which a surface modifying group is attached.

The nanoparticle may be formed from the block copolymer P and a modified polymer P′. The modified polymer P′ is formed by reaction of the block copolymer P with a modified PEG of formula (I).

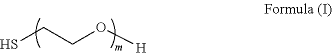

The terminal hydroxyl of the modified PEG of formula (I) reacts with block A of the block copolymer P, cleaving P to form modified polymer P′ that has a terminal sulfhydryl group and a lower molecular weight than P. The terminal “sulfhydryl” may be coupled to a surface modifying group before or after nanoparticle formation by methods known in the art or methods based on those disclosed below.

The coupling agent may be introduced into the block copolymer (or the block copolymer when present in the nanoparticle) by a reversible or irreversible process. In schemes 1-4 below, the term “polymer P” represents the block copolymer before or after formation of a nanoparticle.

In a preferred reversible process, a compound of formula (I) provides a polyalkylene glycol linker and 2,2′-dipyridyl disulfide provides a terminal pyridin-2-yldisulfanyl group that may be reversibly attached to compounds that contain a sulfhydryl group.

Where the block copolymer is terminated with a block A terminated with a diol or diamine group, the compound of formula (II) is reacted with 2,2′-dipyridyl disulfide and the resulting compound is attached to the block copolymer directly. As detailed in Scheme 1 below the block copolymer retains the terminal pyridin-2-yldisulfanyl group.

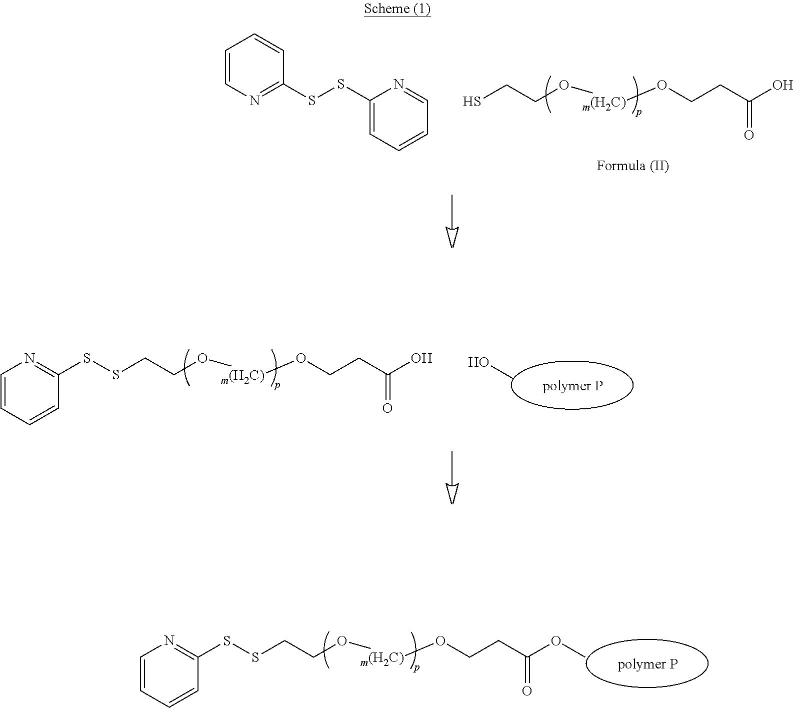

Where the block copolymer is terminated with a block A terminated with a dicarboxylic acid group, the compound of formula (II) is first reacted with a polyalkylene glycol, then modified with a pyridin-2-yldisulfanyl group. It is then attached through the polyalkylene glycol segment to the block copolymer, as detailed in Scheme 2 below wherein the polyalkylene glycol is PEG.

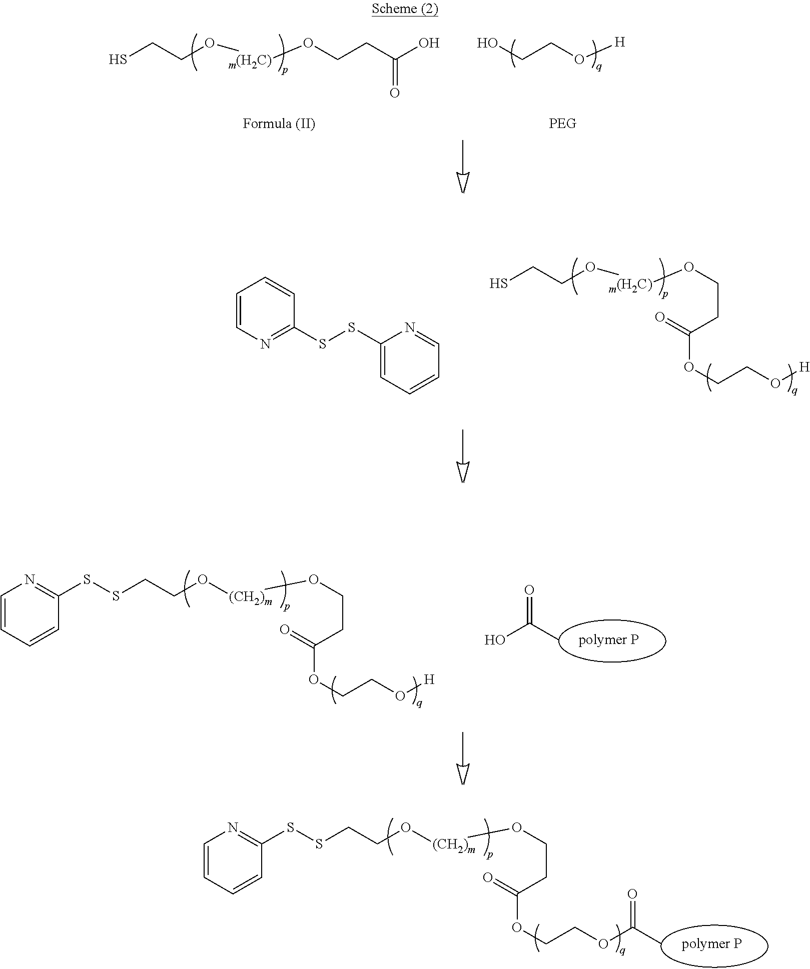

Once the coupling agent has been attached as in scheme 1 or 2, a surface-modifying agent of interest comprising, for instance, a sulfhydryl group is coupled to the block copolymer by reaction with the terminal pyridin-2-yldisulfanyl group to displace pyridine-2-thione.

A preferred irreversible process is based on the polyalkyleneglycolatedcarboxylic acid-carrying maleimide of formula III.

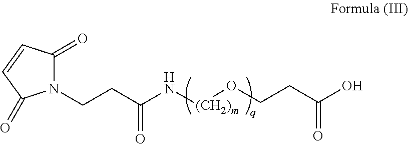

Where the block copolymer is terminated with a block A terminated with a hydroxyl or amino group, the block copolymer may be reacted directly with the compound of formula (III), as exemplified in Scheme 3 below for the hydroxyl-terminated block copolymer.

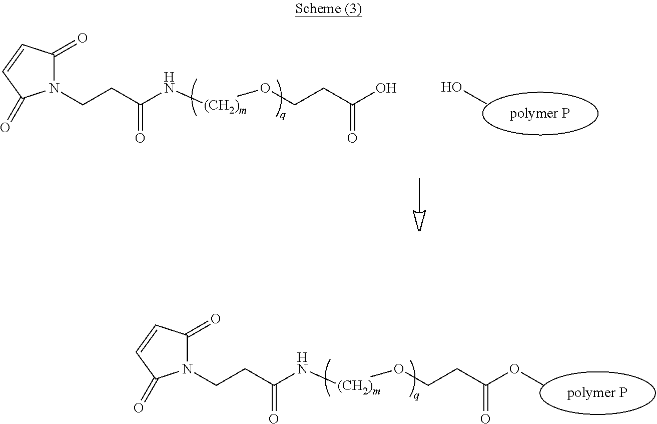

Where the block copolymer is terminated with a block A terminated with a carboxylic group, the reaction proceeds according to the Scheme (4) below. The carboxylic acid moiety on the polyalkyleneglycolated maleimide is activated with N-hydroxysuccinimide and reacted with ethanolamine, before being attached to the block copolymer.

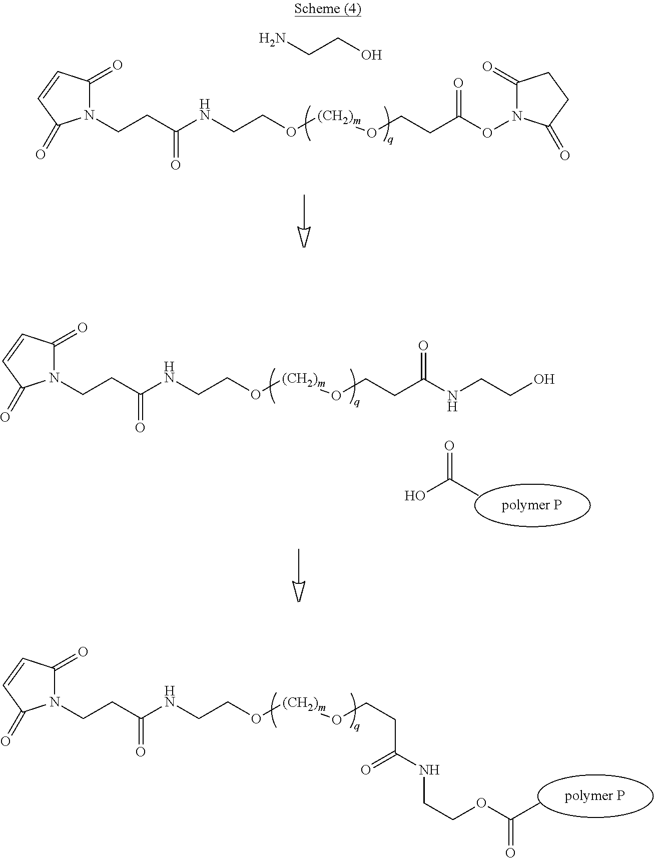

Once the coupling agent has been attached as in scheme 3 or 5, a surface-modifying agent of interest comprising, for instance, a sulfhydryl group is coupled to the block copolymer by reaction with the maleimide carbon-carbon double bond.

In the above schemes, m is a numerical index equal to or greater than 1, preferably from 1 to 8, more preferably from 2 to 5, most preferably 2; p is a numerical index greater than 1, preferably from 2 to 20, more preferably from 4 to 10, most preferably 7; and q is a numerical index greater than 1, preferably from 10 to 450, more preferably from 45 to 70.

The invention therefore provides nanoparticles (NP) that comprise one or more surface-modifying agent(s) connected by all or part of a coupling agent, as shown in scheme 5 below, in which NP represents the nanoparticle comprising the block copolymer with or without, preferably with, one or more active agent(s) incorporated or encapsulated therein and SMA represents one or more surface-modifying agent(s).

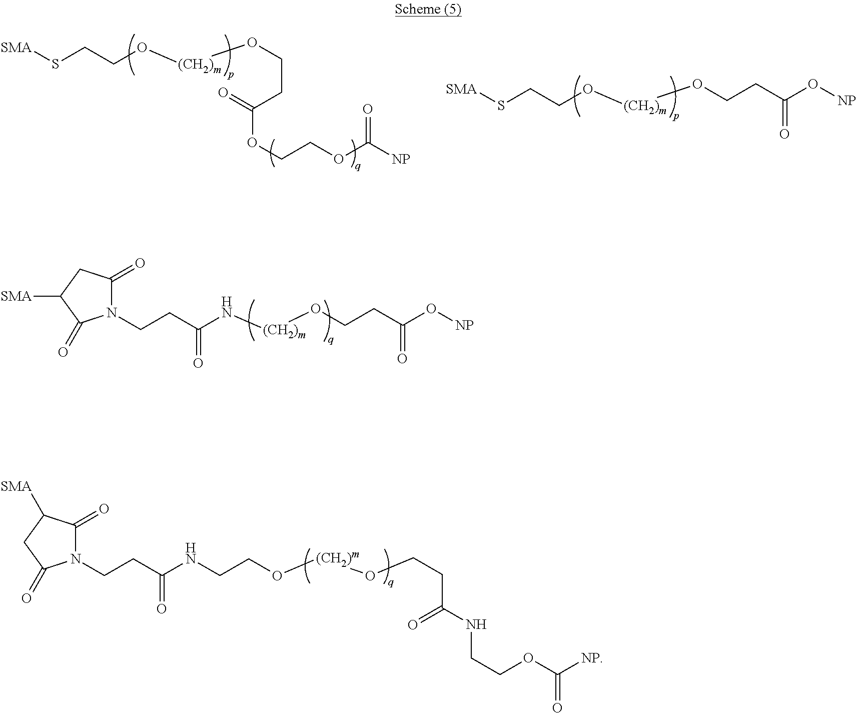

The inventors have further developed a rapid and effective method to modify the surface of the nanoparticles of the present invention, which allows a broader range of surface modifying agents to be associated with the nanoparticles in order to recognize a wider range of targets. The method utilizes a coupling agent comprising a group of Formula (IV), which may be introduced into or onto the nanoparticle via contact with a preformed nanoparticle or with the block copolymer or one of its constituent polymers or monomers prior to nanoparticle formation.

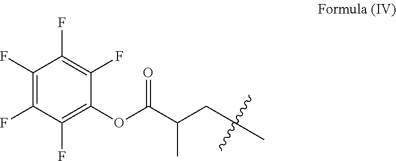 [SMILES: *CC(C)C(=O)Oc1c(F)c(F)c(F)c(F)c1F]

Attachment of the group of Formula (IV) to the nanoparticles of the present invention may be achieved by methods known in the art. In a preferred method, the nanoparticles are treated using cold plasma after a lyophilization step, creating radicals on the surface of the nanoparticles and allowing grafting of the group of Formula (IV) to the surface. Alternatively, the nanoparticles are formed by the emulsion method with a core shell approach. A radical initiator such as persulfate may then be used to allow the groups on the surface of the preformed nanoparticles to react with pentafluorophenyl methacrylate to form groups of Formula (IV) on the shell of the nanoparticle. In another preferred method, at least one of the monomer units B comprises one or more carbon-carbon double bond(s), which may be reacted with pentafluorophenyl methacrylate in order to graft the group of Formula (IV) to the surface of the nanoparticles after their formation.

The group of Formula (IV) provides a reactive ester functionality to facilitate a covalent attachment between the nanoparticle and the surface-modifying agent of interest. In particular, the method may be used to covalently attach surface-modifying groups containing an amine moiety to the nanoparticle. Particularly preferred is surface-modification with thiolated polymers to improve mucosal adhesion, with fluorophors to monitor uptake, or with a BBB signal peptide or an RGD derivative for targeting. Thus, the invention particularly provides nanoparticles and compositions as defined herein wherein the nanoparticles comprise a coupling agent of formula (III) covalently attached thereto.

### BRIEF DESCRIPTION OF THE FIGURES

FIG. 1 shows the steps of synthesis of an exemplary block copolymer of the present invention.

FIG. 2 shows the effect on nanoparticle size of variations in non-solvent (water, methanol, ethanol), solvent:non-solvent ratio (1:20, 1:10, 1:2) and polymer concentration (50 mg/ml, 20 mg/ml and 10 mg/ml).

FIG. 3 shows the formation of a nanoparticle (N) from the block copolymer (P) and the block copolymer to which a surface-modifying agent and, optionally, all or part of a coupling agent has been attached (2P).

FIG. 4 shows the effects of different concentrations of empty nanoparticles (NNP), paclitaxel and paclitaxel-loaded nanoparticles (paclitaxel-NNP) on CGL-1 cells after 14 days colony formation.

FIG. 5 shows the effects of different concentrations of NNP, paclitaxel and paclitaxel NNP on LN-229 cells after 21 days colony formation.

FIG. 6 shows the effects of different concentrations of NNP, paclitaxel and paclitaxel NNP on U-897 MG cells after 14-21 days colony formation.

FIG. 7 shows toxicity of NNP, paclitaxel and paclitaxel NNP to normal human astrocytes (NHA).

FIG. 8 shows toxicity of NNP, paclitaxel in DMSO and paclitaxel NNP to normal human neural progenitors (NHNP).

FIG. 9 shows toxicity of NNP, paclitaxel and paclitaxel NNP to immortalized human neural progenitors (RenCell).

FIG. 10 shows the release profile of paclitaxel from representative nanoparticles of the present invention.

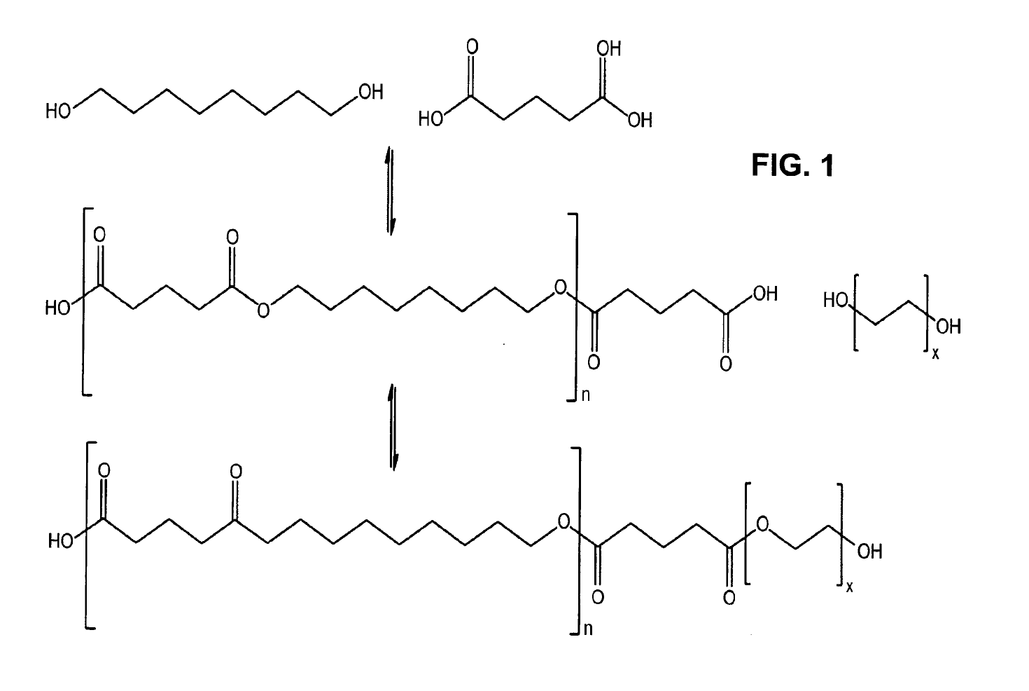

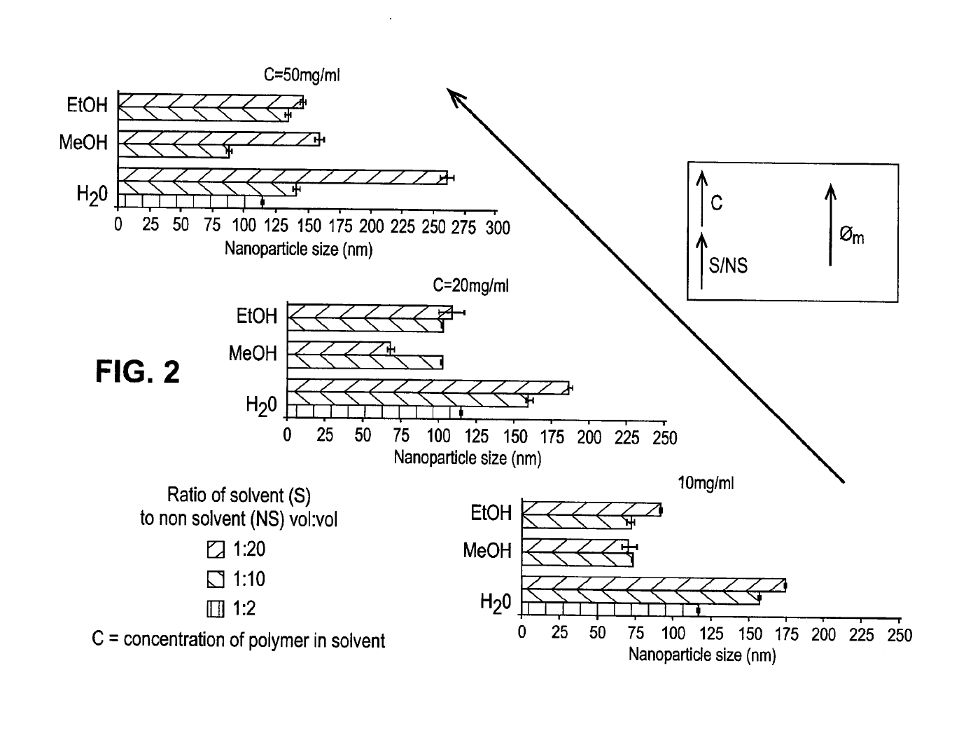

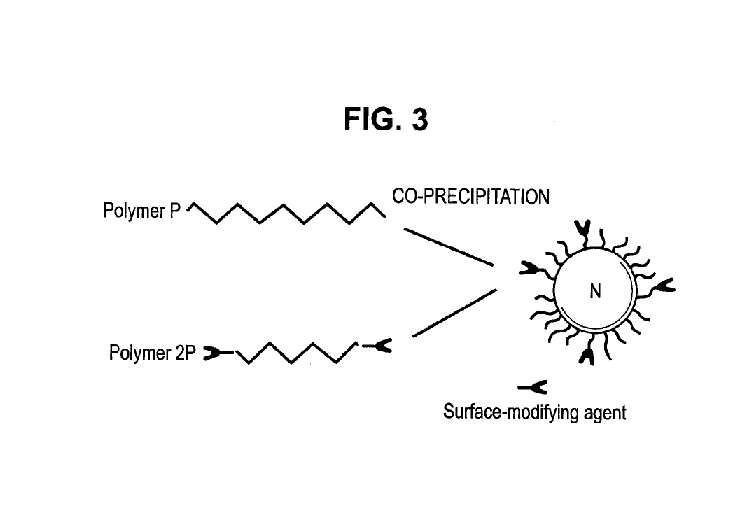

The invention is further illustrated by the following examples. It will be appreciated that the examples are for illustrative purposes only and are not intended to limit the invention as described above. Modification of detail may be made without departing from the scope of the invention.

### EXAMPLES

### Example 1

12 g of glutaric acid (0.09 moles) and 11.1 g of 1,8-octanediol (0.08 moles) are reacted in a microwave oven (Discovery CEM) at a power of 100 W for 1 hour. The work is performed under vacuum (100 mbar) and cooling of the system with compressed air to maintain the temperature constant at 120° C. A rigid block is thus generated.

The rigid block is subsequently reacted with 2000 polyethylene glycol (M_{w} 2000 Da; 6.5 g, 3 mM) in the same microwave reactor for 240 minutes and at a power of 100 W at 120° C., under vacuum and with cooling with compressed air. 10 g of the block biopolymer is thus obtained.

### Example 2

The diffusing medium was acetone in which was dissolved the block copolymer of Example 1 at concentrations of 10, 20 and 50 mg/ml and a quantity of paclitaxel at 3% by weight of the block copolymer. The dispersing medium comprised Milli-Q water, methanol or ethanol.

The diffusing medium was added to the dispersing medium in a ratio of 1:2, 1:10 or 1:20 at a flow rate of 50 μl/min by means of a syringe, controlled by a syringe pump, positioned with the needle directly in the medium, under a magnetic stirring of 130 rpm and at 25° C. The resulting nanosuspension was then centrifuged for 45 min at 6000 rpm in order to gradually remove the dispersing medium, any untrapped paclitaxel and the diffusing medium. The supernatant was discarded and the pellet resuspended in Milli-Q water (15 ml), then centrifuged again under the same conditions in a final washing step. The supernatant is discarded and the pellet may be stored in solution or redispersed in water and lyophilized before storage.

The centrifuged and stored nanoparticles swell, leading to an increase in size until the swelling equilibrium is reached after 5 days in storage. The nanoparticles were characterized after 15 days in storage.

### Example 3

The size and polydispersivity of the nanoparticles produced in Example 2 were analysed by dynamic light scattering using a Zetasizer (Malvern Instruments, UK) at a scattering angle of 90° and at a temperature of 25° C., using samples appropriately diluted with filtered water. The results are shown in Table 2.

TABLE 2

| | | Polymer | Solvent:non- | | |
|---|---|---|---|---|---|
| | Non- | concⁿ | solvent ratio | Size | |
| Ex. | solvent | (mg/ml) | (v:v) | (nm) | Polydispersivity |
| 2.1 | Water | 10 | 1:2 | 116.5 ± 0.9379 | 0.213 ± 0.012 |
| 2.2 | Water | 10 | 1:10 | 157.1 ± 1.278 | 0.178 ± 0.016 |
| 2.3 | Water | 10 | 1:20 | 174.4 ± 1.238 | 0.099 ± 0.015 |
| 2.4 | Water | 20 | 1:2 | 115.4 ± 1.433 | 0.196 ± 0.015 |
| 2.5 | Water | 20 | 1:10 | 159.5 ± 1.612 | 0.197 ± 0.011 |
| 2.6 | Water | 20 | 1:20 | 186.9 ± 1.642 | 0.118 ± 0.015 |
| 2.7 | Water | 50 | 1:2 | 114.6 ± 0.7062 | 0.217 ± 0.009 |
| 2.8 | Water | 50 | 1:10 | 140.8 ± 2.736 | 0.33 ± 0.029 |
| 2.9 | Water | 50 | 1:20 | 261 ± 5.154 | 0.218 ± 0.014 |
| 2.10 | Methanol | 10 | 1:10 | 72.81 ± 0.4731 | 0.098 ± 0.012 |
| 2.11 | Methanol | 10 | 1:20 | 70.52 ± 5.282 | 0.201 ± 0.038 |
| 2.12 | Methanol | 20 | 1:10 | 102.7 ± 0.4787 | 0.106 ± 0.011 |
| 2.14 | Methanol | 20 | 1:20 | 68.43 ± 2.518 | 0.282 ± 0.04 |
| 2.15 | Methanol | 50 | 1:10 | 88.13 ± 2.354 | 0.243 ± 0.034 |
| 2.16 | Methanol | 50 | 1:20 | 159 ± 3.553 | 0.242 ± 0.023 |
| 2.17 | Ethanol | 10 | 1:10 | 71.7 ± 2.779 | 0.204 ± 0.036 |
| 2.18 | Ethanol | 10 | 1:20 | 91.17 ± 0.6609 | 0.131 ± 0.01 |
| 2.19 | Ethanol | 20 | 1:10 | 102.7 ± 0.4787 | 0.106 ± 0.011 |
| 2.20 | Ethanol | 20 | 1:20 | 108.9 ± 8.399 | 0.236 ± 0.036 |
| 2.21 | Ethanol | 50 | 1:10 | 134.8 ± 1.7 | 0.218 ± 0.021 |
| 2.22 | Ethanol | 50 | 1:20 | 146.2 ± 1.5 | 0.207 ± 0.014 |

### Example 4

The zeta-potential of the nanoparticles produced in Example 2.6 were analysed with an electrophoresis analyser setup, with a Smoluchowsky constant of 1.5 to achieve zeta-potential values from electrophoretic mobility. The zeta-potential was found to be in a range of −35-−40 mV.

### Example 5

The active agent encapsulation efficiency, active agent entrapment, active agent release profile and kinetic degradation profile of the nanoparticles produced in Example 2 were determined by HPLC analysis with a reverse-phase C-18 column and eluted isocratically with acetonitrile/water (70/30 v/v), The flow rate fixed at 1 ml/min and detection obtained by UV detection at 227 nm. Table 3 shows active agent encapsulation efficiency and active agent entrapment data.

TABLE 3

| | |
|---|---|
| Amount of polymer (mg) | 14.25 |
| Amount of active agent (mg) | 0.4275 |
| Theoretical active agent entrapment (% w/w) | 2.91% |
| Active agent encapsulated (mg) | 0.305 |
| Active agent not encapsulated (mg) | 0.0843 |
| Encapsulation efficiency (%) | 71.3 |
| Active agent entrapment (%) | 2.09 |
| Active agent lost (mg) (%) | 0.0382 (9%) |

### Example 6.1

### Cytotoxicity in Glioma Cells

A clonogenic assay was carried out to observe the toxicity of decorated paclitaxel-loaded nanoparticles compared with that of paclitaxel and empty nanoparticles on glioma cell lines and determine IC₅₀ values in a long term effect (2 to 3 weeks growth). Nanoparticles with or without a surface-modifying agent (SEQ ID#5) were formed according to the method of example 2, with or without inclusion of paclitaxel.

Three cells lines were used. The CGL-1 cell line (Oncodesign, Dijon, France) was isolated from the TG-1 tumour subcutaneously (SC) implanted in Nude rat. 14 days were allowed for colony formation. The human U-87 MG cell line (American Type Culture Collection) was initiated from a grade III glioblastoma from a 44 year old female Caucasian. 21 days were allowed for colony formation. Finally, the LN-229 cell line (American Type Culture Collection) was established in 1979 from cells taken from a patient with right frontal parieto-occipital glioblastoma. 14-21 days were allowed for colony formation.

The formulations tested were as follows: nanoparticles (stock solution NaCl 0.9%); paclitaxel-loaded nanoparticles (3.33% paclitaxel by weight; stock solution NaCl 0.9%); and paclitaxel (stock solution DMSO 100%). All test substances diluted at 100 μM in their respective vehicle to obtain stock solutions. Five concentrations (1:5 or 1:3 dilution steps) were used in triplicate. Formulations were obtained by diluting stock solutions at 100 μM in their respective vehicle to obtain a series of five concentrations in 1:5 or 1:3 dilution steps. Each solution was then further diluted at 1:20 with RPMI 1640 before final dilution at 1:10 into soft agar.

The initial concentrations tested were 0.8 nM, 4 nM, 20 nM, 100 nM and 500 nM. Repetitions were carried out for GCL-1 at 2 nM, 8 nM, 40 nM, 200 nM and 1000 nM and for LN-229 at 1.2 nM, 3.7 nM, 11 nM, and 33 nM. At least 2 independent experiments were carried out with top concentrations and dilution steps changed when needed. Cells were incubated for 14 to 21 days with the different treatments.

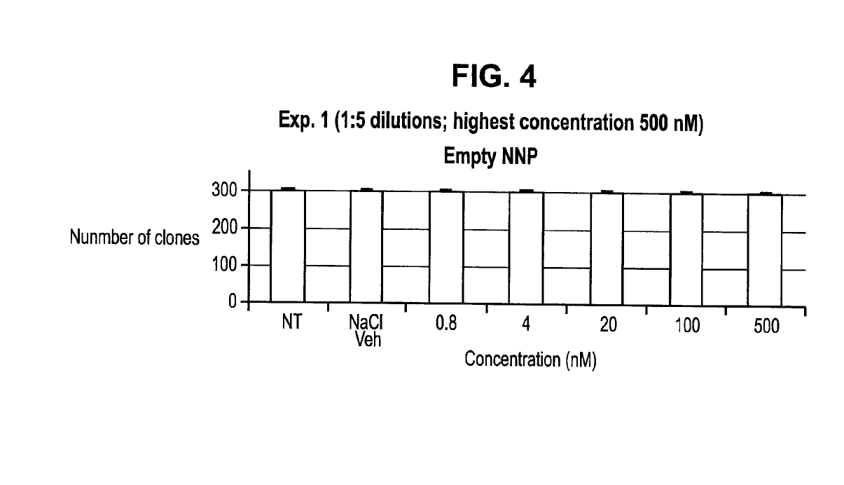

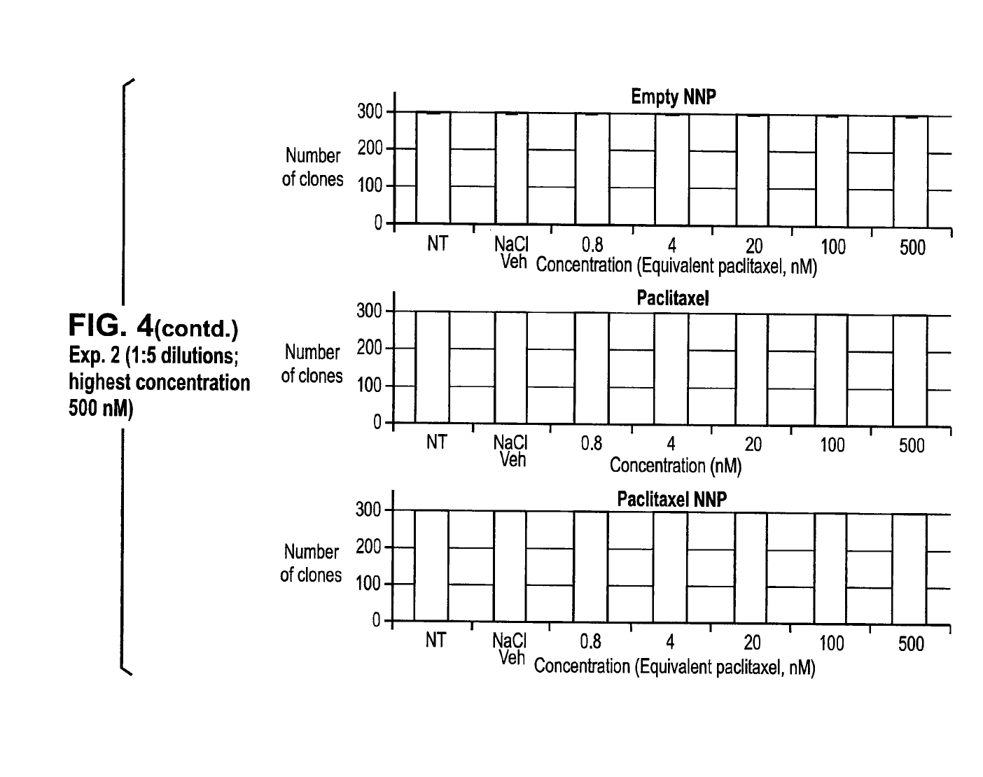

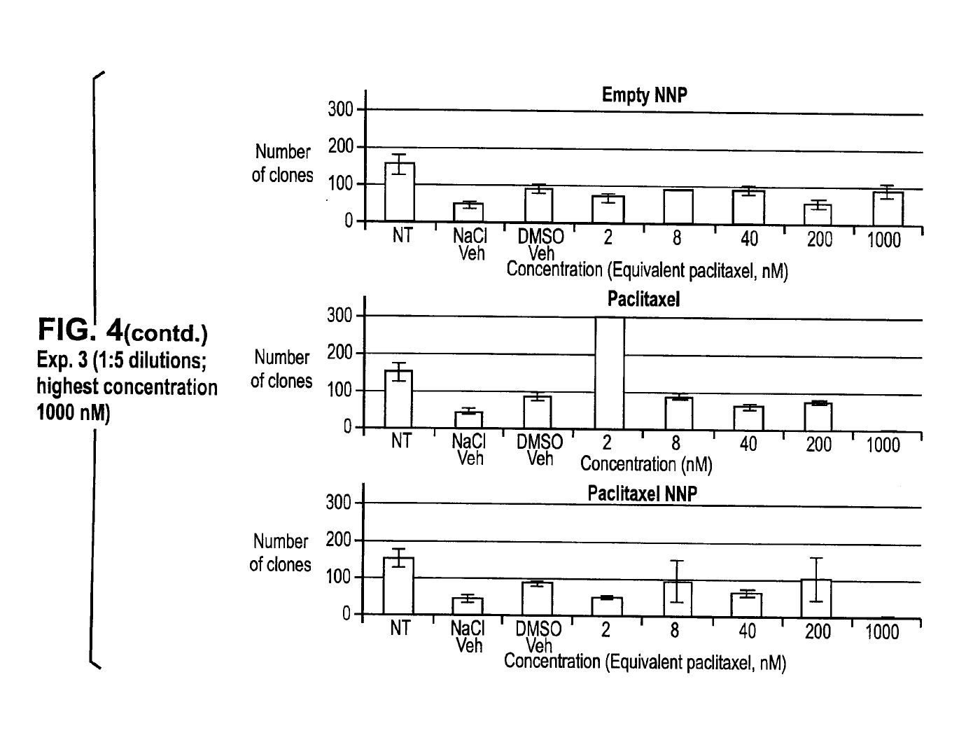

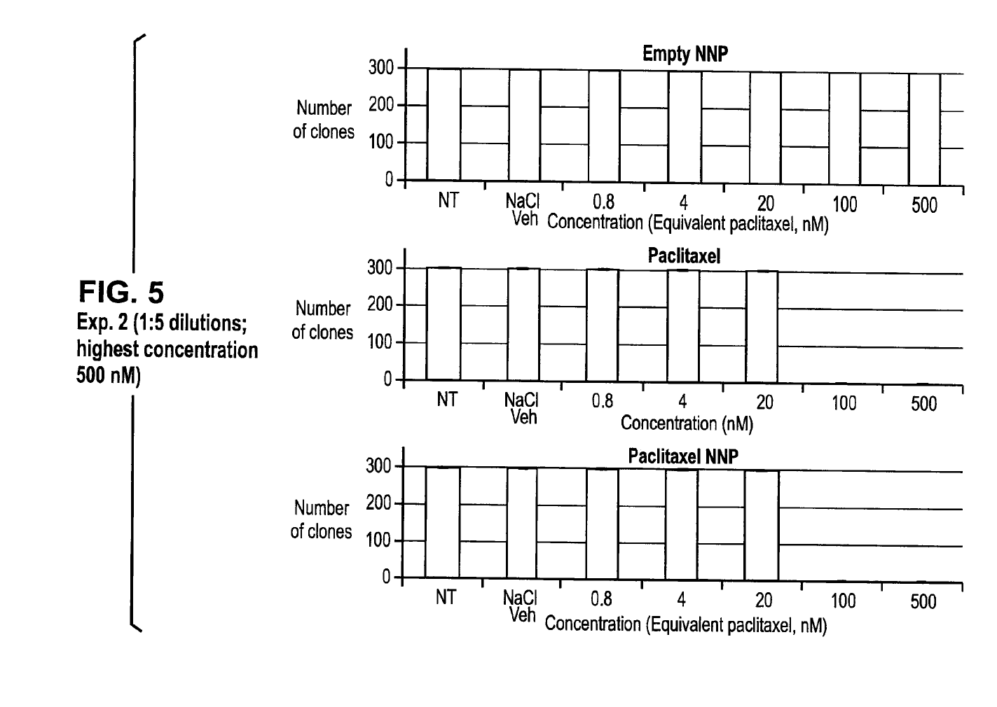

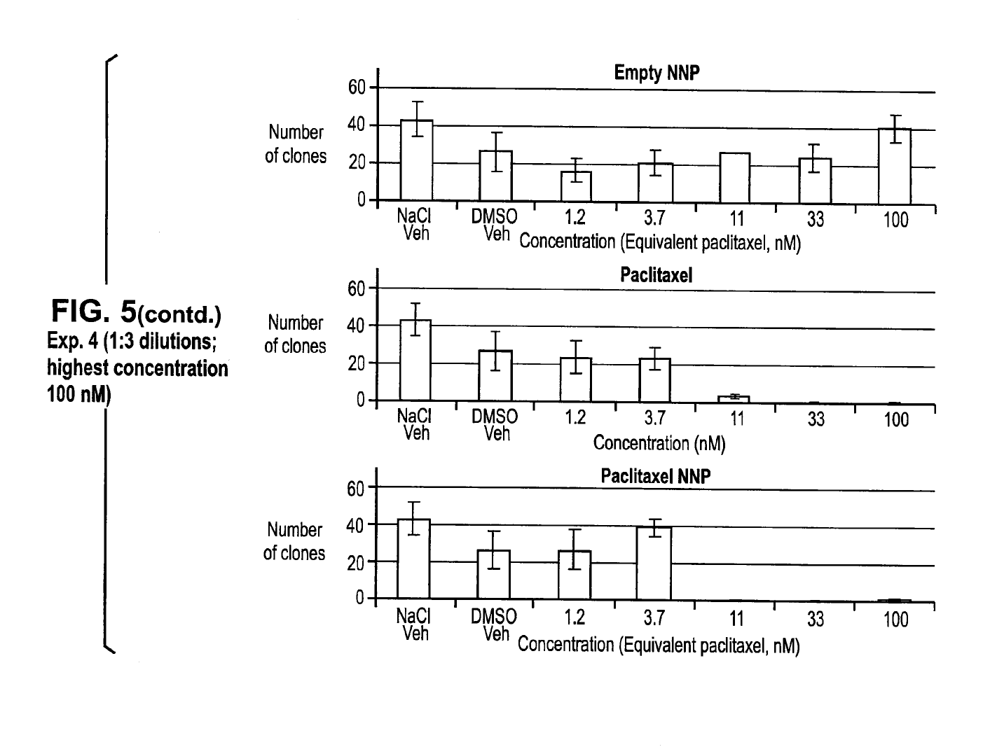

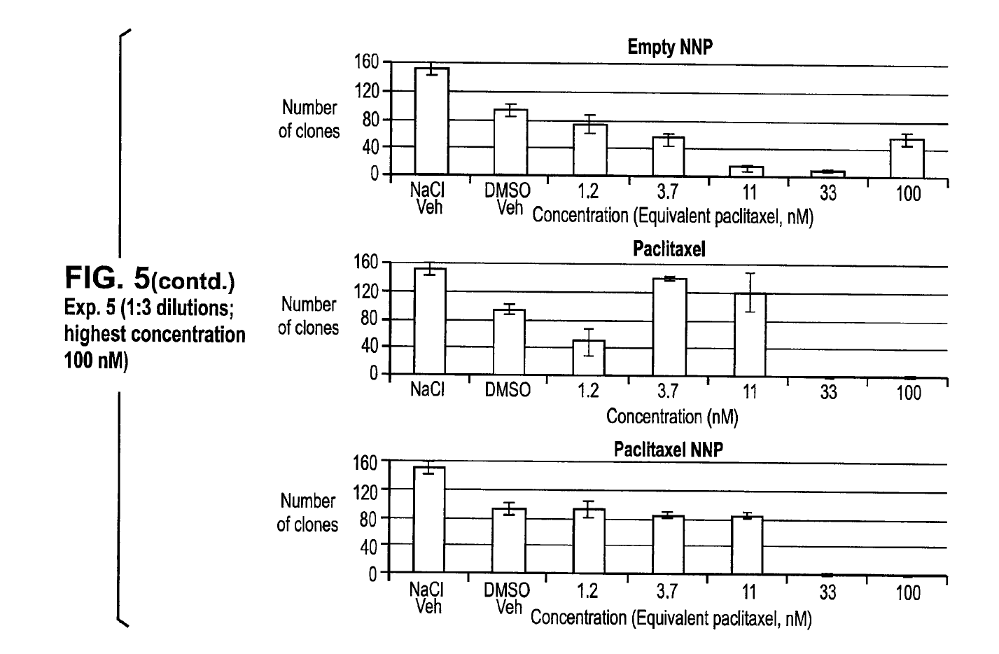

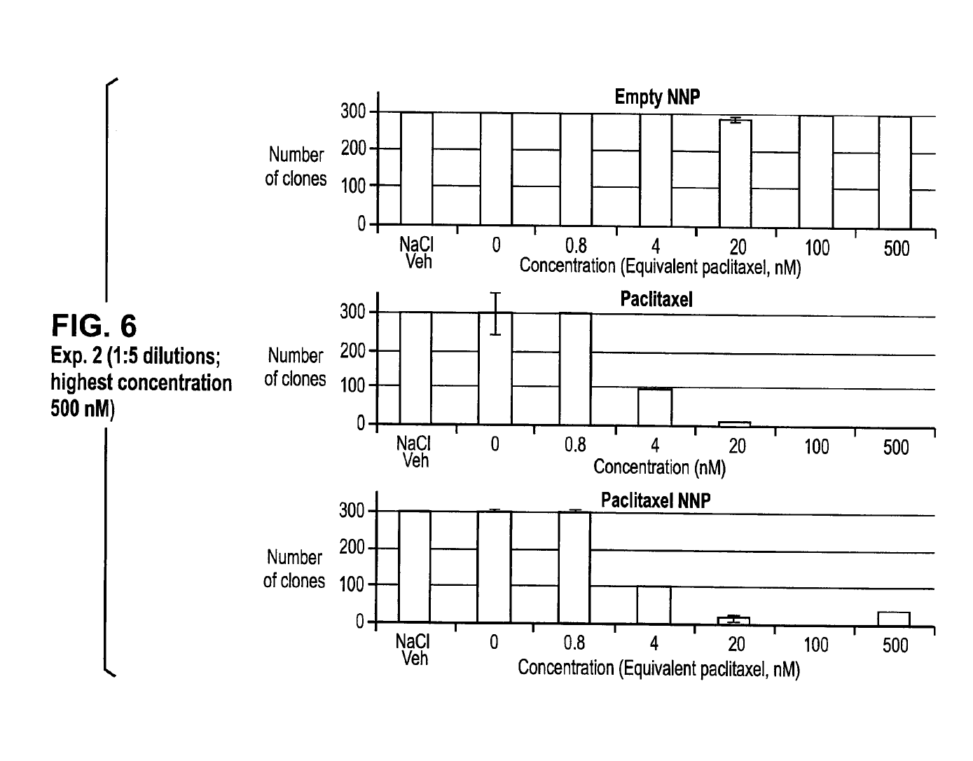

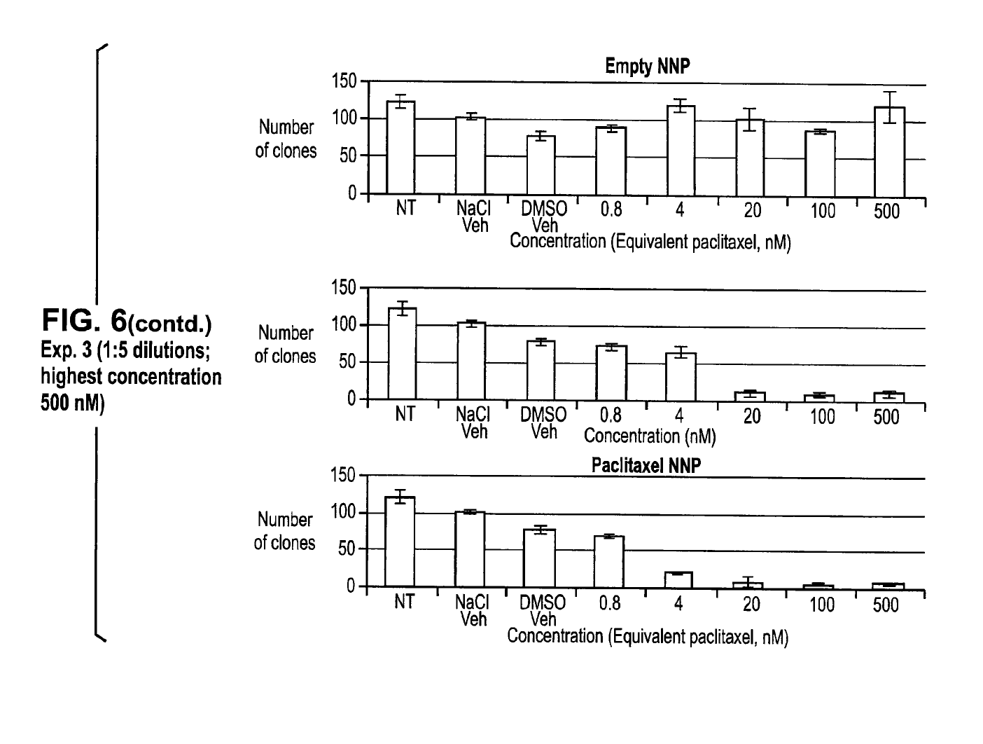

Results are given in Table 4 and indicate the % survival from the initial 300 clones. Vehicle results not included (100% survival in all cases). The results are shown graphically in FIGS. 4, 5 and 6. Clonogenic tests are based on clones of cells and not cells alone. Therefore, the IC₅₀ values given correspond to concentration that inhibits 50% of clones.

TABLE 4

| Cell line | CGL1 | LN229 | | U87-MG | |
|---|---|---|---|---|---|
| Experiment number | 3 | 4 | 5 | 2 | 3 |
| Serial dilutions | 1:5 | 1:3 | 1:3 | 1:5 | 1:5 |
| Highest concentration | 1000 | 100 | 100 | 500 | 500 |
| tested (nM) | | | | | |
| Empty nanoparticles | >1000 | >100 | >100 | >500 | >500 |
| (IC₅₀; nM) | | | | | |
| Paclitaxel (IC₅₀; nM) | 792 | 7.3 | 21 | 2.6 | 7.5 |
| Paclitaxel nanoparticles | 937 | 8.7 | 14 | 2.3 | 1.1 |
| (IC₅₀; nM) | | | | | |

| | | | |
|---|---|---|---|
| Paclitaxel metabolic assay | >100 | Not performed | ~10 |
| (historical data) (IC₅₀; nM) | | | |

For each of the three tumour cell lines the empty nanoparticles showed little or no cytotoxicity, and the paclitaxel-loaded nanoparticle showed similar or higher cytotoxicity compared with paclitaxel alone. Therefore, the nanoparticle does not reduce paclitaxel activity. The difference of activities between the three tumour cell lines correlated to the IC₅₀ observed when cells are treated for few days and assayed with metabolic assays.

The results indicate that paclitaxel-loaded nanoparticles are as effective as paclitaxel and that empty nanoparticles are non-toxic to cancer cells. The IC₅₀ for U87-MG is between 1.1-2.3 nM, which is lower than literature values for paclitaxel alone (10-20 nM).

The test was also repeated for U87-MG cells, showing a superiority trend for the loaded nanoparticles versus paclitaxel alone (IC₅₀ values of 0.8-4 nM versus 4-20 nM respectively).

### Example 6.2

### Cytotoxicity in Normal Neuronal Cells

An ATP-lite assay was carried out over 48 to 72 hours in order to determine cytotoxicity of paclitaxel-loaded nanoparticles comprising a surface-modifying agent compared with that of paclitaxel and empty nanoparticles on healthy brain cell lines and determine IC₅₀ values.

The formulations tested were as follows: vehicle, empty nanoparticles, decorated paclitaxel-loaded nanoparticles (3.33% paclitaxel by weight; decoration: SEQ ID #5) paclitaxel and etoposide (etoposide is described as having mild toxicity in treatment of brain cancer).

The concentrations tested were 0.00026 nM, 0.0013 nM, 0.0064 nM, 0.032 nM, 0.16 nM, 0.8 nM, 4 nM, 20 nM, 100 nM and 500 nM. Etoposide at 50 μM

Three cell lines were tested. Normal human astrocytes (NHA; Lonza) are primary-derived cultures of adherent cells with limited number of divisions. Normal human normal progenitors (NHNP; primary cell line; Lonza) are neurosphere growing cells with high number of division that differentiate in adherent glioma cells and neurons under specific conditions (laminin coated plates, induction with differentiation factors). Finally, immortalized human neural progenitors (RenCells; Millipore) are fetal brain cells transformed with c-myc oncogene.

Cells were incubated with treatment for 24 hours (astrocytes) and 72 hours (progenitor cell lines)

### 1) Astrocytes

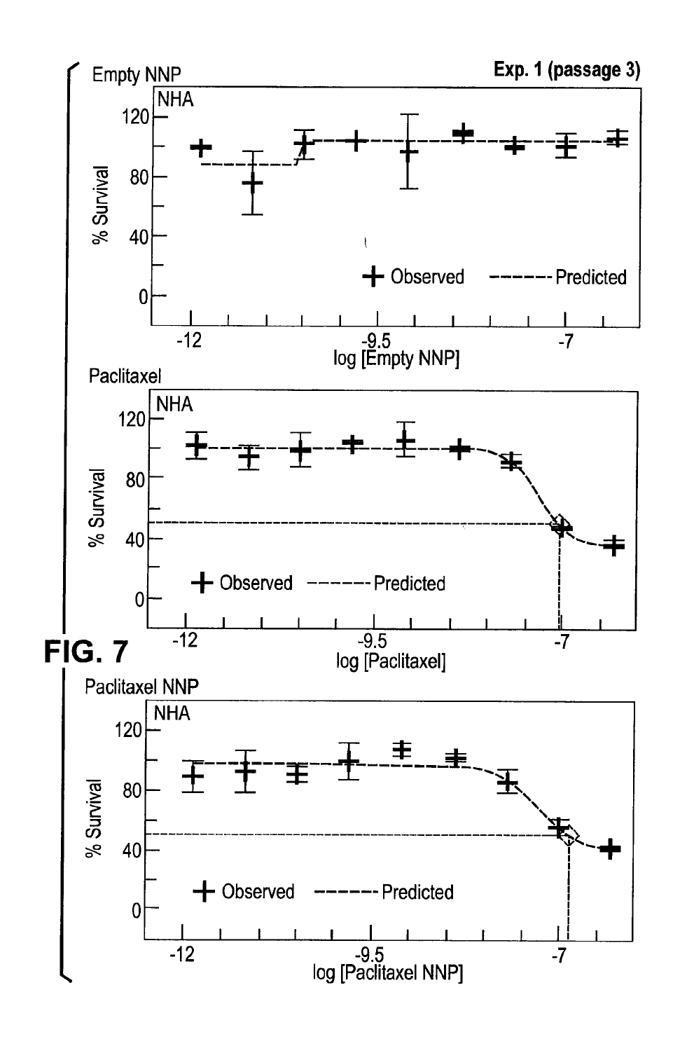

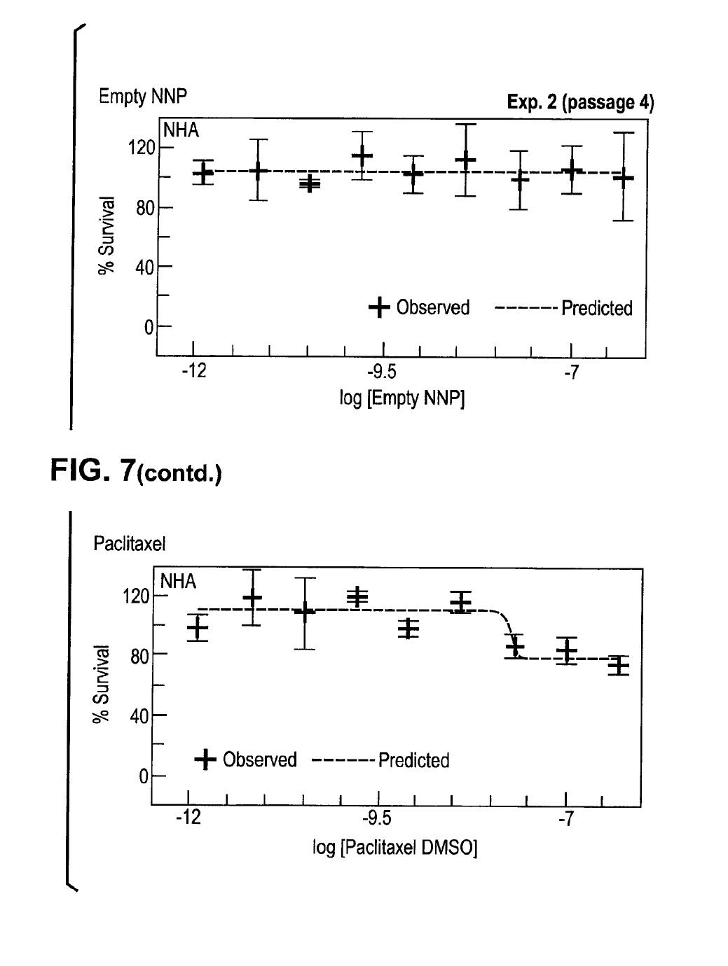

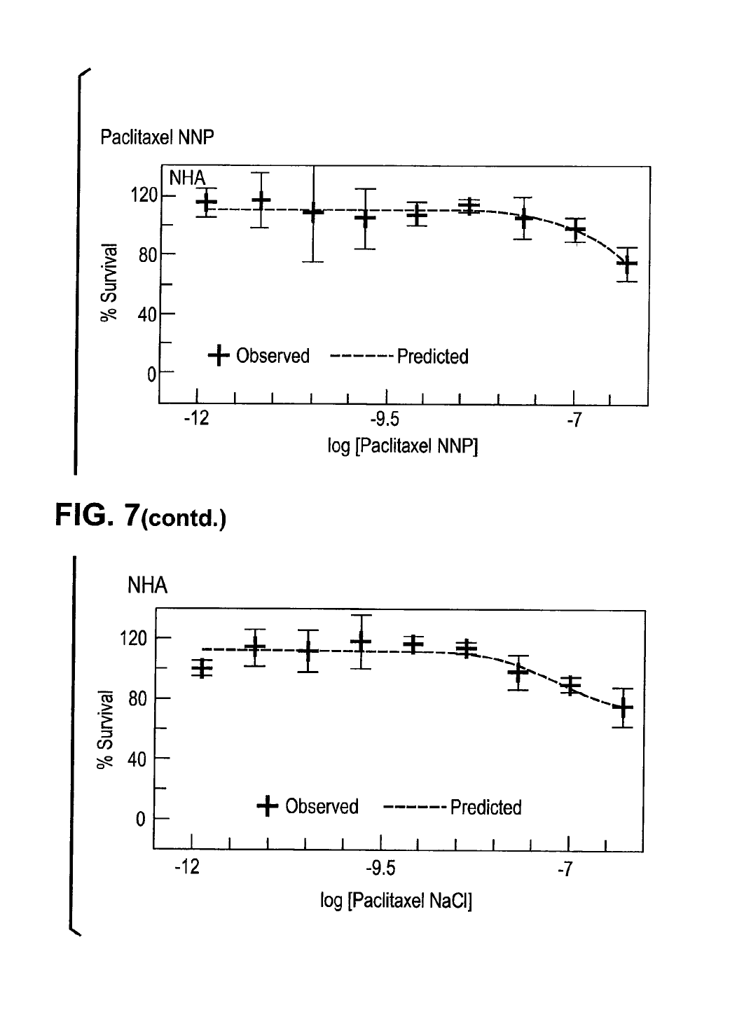

The results are shown in FIG. 7. Empty nanoparticles are non-toxic in the whole range of concentrations tested, thus IC₅₀ >500 nM. Paclitaxel and decorated paclitaxel-loaded nanoparticles showed similar toxicity, with IC₅₀ values of about 100 nM. At 50 μM, only 12% of cells treated with etoposide survived.

The experiment was repeated using saline as vehicle for paclitaxel (instead of DMSO:saline; results shown in FIG. 7). This again showed that empty nanoparticles lacked toxicity in all the ranges studied and showed a less pronounced toxicity for the paclitaxel-loaded nanoparticles and paclitaxel alone, resulting in IC₅₀ >500 nM. Etoposide survival rate was 45%, thus showing an IC₅₀ around 50 μM.

### 2) Normal Human Neural Progenitors

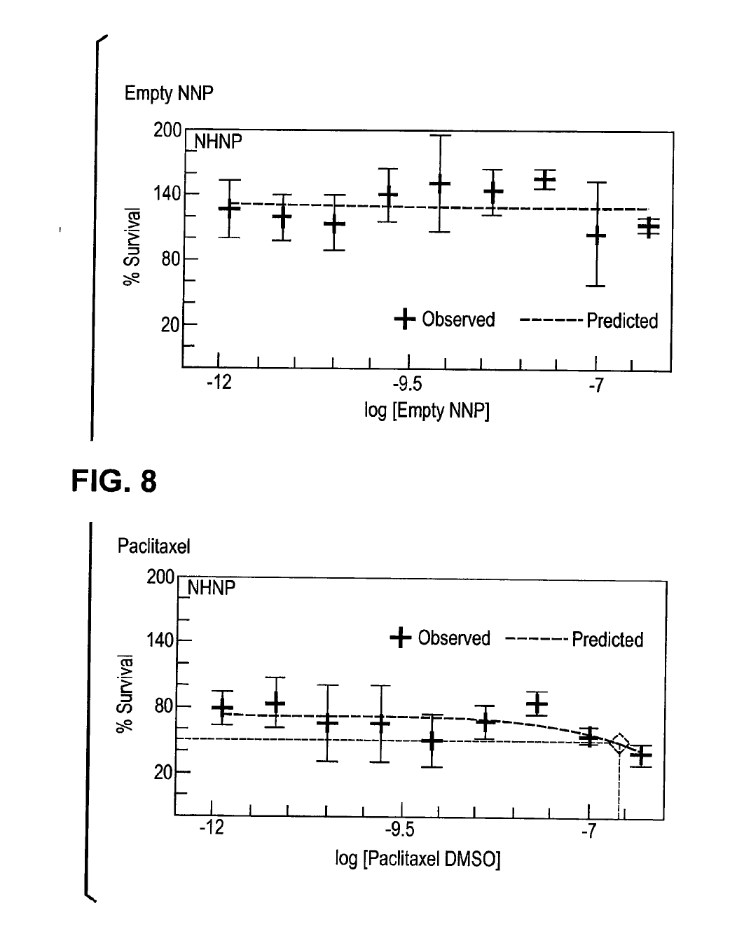

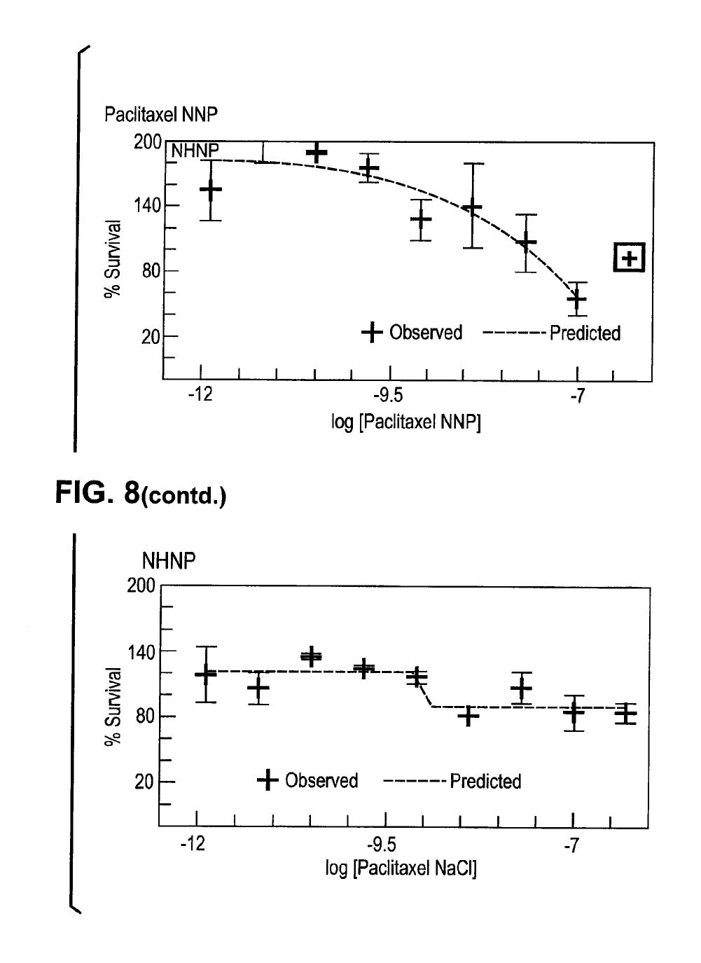

The results are shown in FIG. 8. Again, empty nanoparticles were not toxic throughout the tested range. Paclitaxel-loaded nanoparticles showed a slight tendency to increase toxicity with concentrations though showing IC₅₀ >500 nM. Paclitaxel dissolved in DMSO:saline showed IC₅₀ between 100-500 nM and >500 nM in saline. Etoposide behaved similarly to tests in astrocytes, showing 21% survival rate at 50 μM.

### 3) Immortalized Human Neural Progenitors (ReNcells)

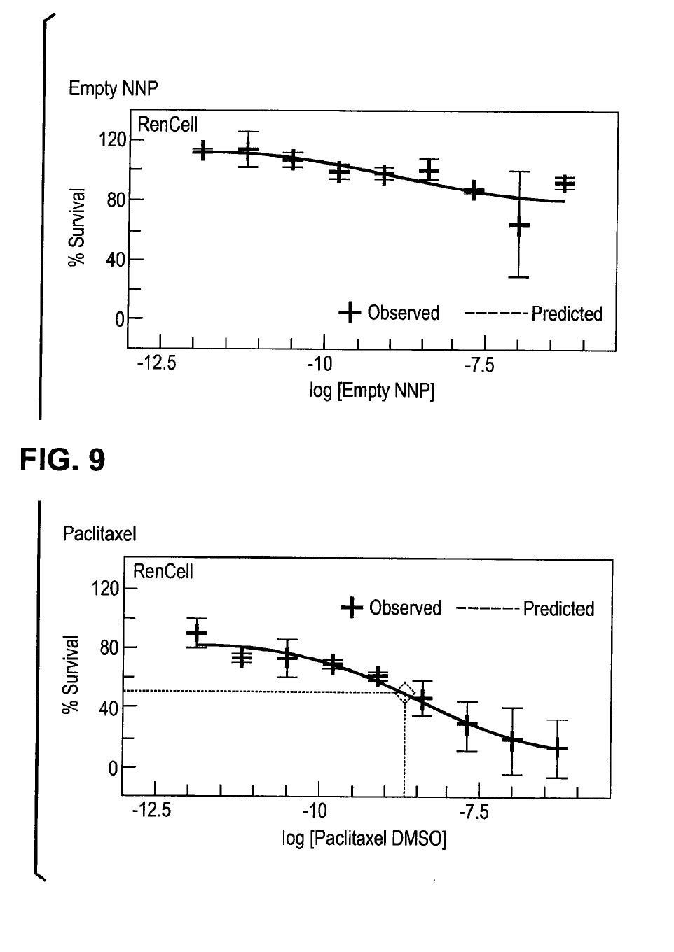

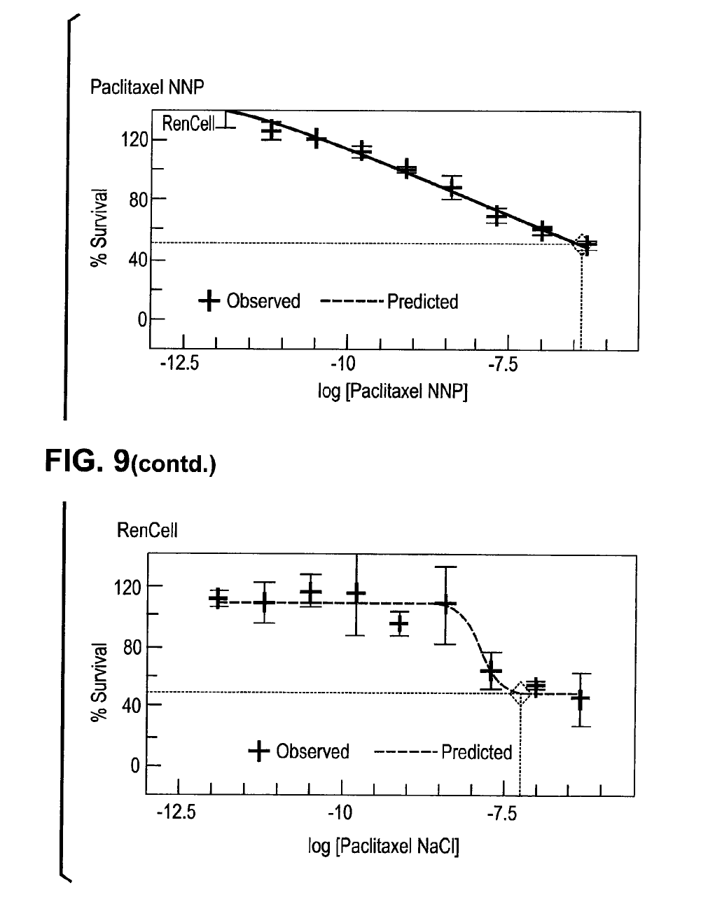

The results are shown in FIG. 9. Again, empty nanoparticles were not toxic throughout all the tested range. Paclitaxel-loaded nanoparticles showed some tendency to increase toxicity with concentrations with an IC₅₀ around 500 nM. Paclitaxel dissolved in DMSO:saline showed IC₅₀ around 2 nM and 57 nM in saline. Etoposide showed 3% survival rate at 50 μM.

A summary of IC₅₀ data is provided in Table 6. No toxicity was observed with empty nanoparticles at the tested concentrations. The IC₅₀ values of paclitaxel-loaded nanoparticles are higher than those of paclitaxel alone. This may be because the contact time with the nanoparticles was no longer than 78 hours, and therefore the nanoparticles released only a small percentage of the contained paclitaxel, which causes some degree of toxicity when administered alone in the experiment. This indicates that the nanoparticles have sustained release behaviour.

TABLE 5

| | | | Normal | Immortalized |
|---|---|---|---|---|
| | Normal human | | human | human |
| Cell | astrocytes | | neuron | neural |
| line | Exp. 1 | Exp 2 | progenitors | progenitors |
| Empty NNP (IC₅₀; nM) | >500 | >500 | >500 | >500 |
| Paclitaxel (IC₅₀; nM) | 90 | >500 | 254 | 2.2 |
| Paclitaxel NNP (IC₅₀; nM) | 135 | >500 | >500 | 413 |
| Etoposide (% survival) | 13 | 45 | 21 | 3 |

### Example 7

### Observation of the In Vivo Activity of Paclitaxel-Loaded Nanoparticles on a Glioma Tumour Model in Rat

### Test Materials

TABLE 6

| | Empty | | | |
|---|---|---|---|---|
| | nanoparticle | Loaded nanoparticle | | |
| Batch | SAG005-113/50 | SAG005-122/12.5 | SAG005-122/25 | SAG005-122/50 |
| Amount sent (mg) | 422.4 | 119.8 | 223 | 447.4 * |
| NP weight (mg) | 249.9 | 66.56 | 123.89 | 248.56 * |
| Particle size (nm) | 271.8 ± 0.5 | 241.6 ± 3.1 | 244.4 ± 2.1 | 244.2 ± 2.1 |
| PDI | 0.27 ± 0.02 | 0.31 ± 0.03 | 0.28 ± 0.02 | 0.28 ± 0.04 |
| Surface charge (mV) | −40.3 ± 0.7 | −34.9 ± 0.9 | −36.6 ± 1.5 | −36.9 ± 0.3 |
| Osmolarity (Osm/kg) | 297 | 281 | 285 | 288 |
| pH | ~5 | ~5 | ~5 | ~5 |
| Endotoxin free | Yes | Yes | Yes | Yes |
| Drug description | ***(Not-Loaded)*** | Paclitaxel | Paclitaxel | Paclitaxel |
| Drug content | ***(Not-Loaded)*** | 4.55% of polymeric | 4.55% of polymeric | 4.55% of polymeric |
| | | nanoparticles weight | nanoparticles weight | nanoparticles weight |
| | | (2.53% of the total weight) | (2.53% of the total weight) | (2.53% of the total weight) |

Each vial is reconstituted with the amount of water for injection (wfi, Aguettant) indicated in Table 7.

TABLE 7

| | Empty | | | |
|---|---|---|---|---|
| | nanoparticle | Loaded nanoparticle | | |
| Batch | SAG005- | SAG005- | SAG005- | SAG005- |
| | 113/50 | 122/12.5 | 122/25 | 122/50 |
| Volume of wfi needed | 5 | 5.33 | 4.96 | 4.86 |
| for reconstitution (ml) | | | | |
| Nanoparticle final | 50 | 12.5 | 25 | 50 |
| concentration (mg/ml) | | | | |
| Paclitaxel final | 0 | 0.57 | 1.14 | 2.27 |
| concentration (mg/ml) | | | | |

Following reconstitution, the solution is vortexed for a few seconds and sonicated for 30 minutes (Frequency: 50/60 Hz, Power: 360 W). The particle dispersion (a milky liquid) is then ready for injection. At the time of injection, the samples are filtered with a 0.45 μm filter (equivalent to Millipore Millex HV-Durapore PVDF Membrane).

### Definition of Acute Toxicity: Maximum Tolerated Dose (MTD) Determination

Rats were randomized based on body weight (4 groups, 3 rats/group, 12 rats in total). The active agent-loaded nanoparticle composition was prepared at 5, 10 and 20 mg/kg/injection. The nanoparticle used for the study was freeze-dried, isotonic and could be filtered with no difficulty through a 0.45 micron mesh.

TABLE 8

| | Animals | | Nanoparticles | Paclitaxel | | |
|---|---|---|---|---|---|---|
| Group | (n) | Treatment | (mg/kg/inj) | (mg/kg/inj) | Route | Treatment Schedule |
| 1 | 3 | Vehicle (empty | 440 | — | IV | Q1Dx1 |
| | | particle) | | | | |
| 2 | 3 | Active agent | 110 | 5 | IV | Q1Dx1 |
| | | loaded nanoparticle | | | | |
| 3 | 3 | Active agent | 220 | 10 | IV | Q1Dx1 |
| | | loaded nanoparticle | | | | |
| 4 | 3 | Active agent | 440 | 20 | IV | Q1Dx1 |
| | | loaded nanoparticle | | | | |
| Total | 12 | | | | | |

IV: intravenous injection; Q1D: once daily.

Rat body weight was monitored twice weekly. Rat behaviour and survival was monitored daily. No side effects were detected and rats did not lose weight. In some cases, weight gain was observed. Sacrifice and autopsy (macroscopic examination) of surviving rats was carried out 14 days after treatment. The rats were tested for macroscopic changes in organs. None were observed.

These results indicate that the nanoparticles are non-toxic to animals and, since no toxicity was found at highest dose, the results may indicate sustained release profile. In principle, if paclitaxel alone had been injected at the same doses, side effects should have been observed, especially at highest dose.

At the equivalent highest concentration tested (50 mg of nanoparticle/ml, drug content 4.4%), and assuming blood-brain passage of <1% and a sustained release profile of around 2 weeks, it is predicted that such formulations can be given to humans in a small volume (200 ml) in order to achieve brain concentrations much higher than the IC₅₀.

### Definition of Treatment Toxicity: Maximum Total Tolerated Dose (MTTD) Determination

Rats are randomized based on body weight (4 groups, 3 rats/group, 12 rats in total). The active agent-loaded nanoparticle composition to be tested is prepared at 3 doses.

TABLE 9

| | Animals | | | | Treatment |
|---|---|---|---|---|---|
| Group | (n) | Treatment | | Route | schedule |
| 1 | 3 | Vehicle (empty particle) | | IV | TW x 4 |
| 2 | 3 | The active agent-loaded | Dose #1 | IV | TW x 4 |
| | | nanoparticle composition | | | |
| 3 | 3 | The active agent-loaded | Dose #2 | IV | TW x 4 |
| | | nanoparticle composition | | | |
| 4 | 3 | The active agent-loaded | Dose #3 | IV | TW x 4 |
| | | nanoparticle composition | | | |

| | |
|---|---|
| TOTAL | 12 |

IV: intravenous injection; TW x 4: Twice weekly for 4 consecutive weeks.

Rat body weight is monitored twice weekly. Rat behaviour and survival is monitored daily. Sacrifice and autopsy (macroscopic examination) of surviving rats is carried out 7 days after treatment. If all of the tested doses are toxic, lower doses are tested in additional rats. Once MTTD is defined, an antitumour activity study may be performed in Nude rats bearing the orthotopic U-87 MG tumour model.

### Antitumor Activity Study:

The U-87 MG human glioma cell line is amplified in vitro. 44 female Nude rats are irradiated. Orthotopic injection of U-87 MG human glioma cells in the brain of the rats is then carried out. Following IV injection of Gd-DTPA contrast agent into the tail vein of all rats under anaesthesia at 1 timepoint, MRI analysis is carried out to assess tumour morphology (44 rats, 44 scans). The resulting images are analysed to determine tumour volume. Rats are randomized based on body weight and tumour volume (5 groups, 8 rats/groups, 40 rats). The test substance is prepared at 3 doses and temozolomide is prepared at 50 mg/kg/injection).

TABLE 10

| | No. | | | Dose | Treatment |
|---|---|---|---|---|---|
| Group | Animals | Treatment | Route | (mg/kg/adm) | Schedule |
| 1 | 8 | Vehicle | IP | — | TW x4 |
| | | (empty particle) | | | |
| 2 | 8 | Test substance | IP | Dose #1 | TW x4 |
| 3 | 8 | Test substance | IP | Dose #2 | TW x 4 |
| 4 | 8 | Test substance | IP | Dose #3 | TW x 4 |
| 5 | 8 | Temozolomide | PO | 50 | (Q1Dx5) x2 |

| | |
|---|---|
| TOTAL | 40 |

IP: intraperitoneal injection; PO: per os; TW x 4: twice weekly for 4 consecutive weeks.

Rat body weight is monitored twice weekly. Rat behaviour and survival is monitored daily. MRI analysis for tumour morphology is carried out following IV injection of the Gd-DTPA contrast agent into the tail vein of all rats under anesthesia at two timepoints (8 rats/group/timepoint, 5 groups, 2 timepoints, 80 scans). The resulting images are analysed to determine tumour volume. Sacrifice and autopsy (macroscopic examination) of all rats is carried out after a maximum of 2 months. The paclitaxel level in tumour and brain samples may be quantified by HPLC-MS/MS.

### Pharmacokinetic and Biodistribution of Drug-Loaded Nanoparticles in Nude Rats

Thirty-eight (38) Nude rats are randomized into 1 group of 3 rats and 7 groups of 5 rats according to their individual body weight. The mean body weight of each group is not different from the others (analysis of variance). The monitoring of rats is performed as described above.
Group 1: Three (3) rats are not treated,
Groups 2 to 8: Thirty-five (35) rats receive one IV injection of paclitaxel-loaded nanoparticle at MTD (Q1Dx1) and are sacrificed at different time points (T1 to T7) by cardiac puncture from the different groups under anaesthesia.

Total blood is collected into Capiject® capillary blood collection tubes containing lithium-heparin as anticoagulant (Ref. T-MLHG, Terumo) thoroughly mixed and centrifuged at 2500 rpm for 10 minutes at +4° C. The resulting plasma is collected, separated in five aliquots and stored at −80° C. until analysis. Brains are collected and cut into two parts. The samples are transferred in a dry plastic tube that are immediately snap frozen (in liquid nitrogen) and stored at −80° C. until analysis. All animals are autopsied by macroscopic examination.

Paclitaxel levels in injected solutions, plasma samples and brain samples are determined. The analytical procedure for the determination of paclitaxel in rat samples involves the extraction of the analytes from plasma and HPLC/MS-MS analysis using docetaxel as an internal standard.

### Example 8

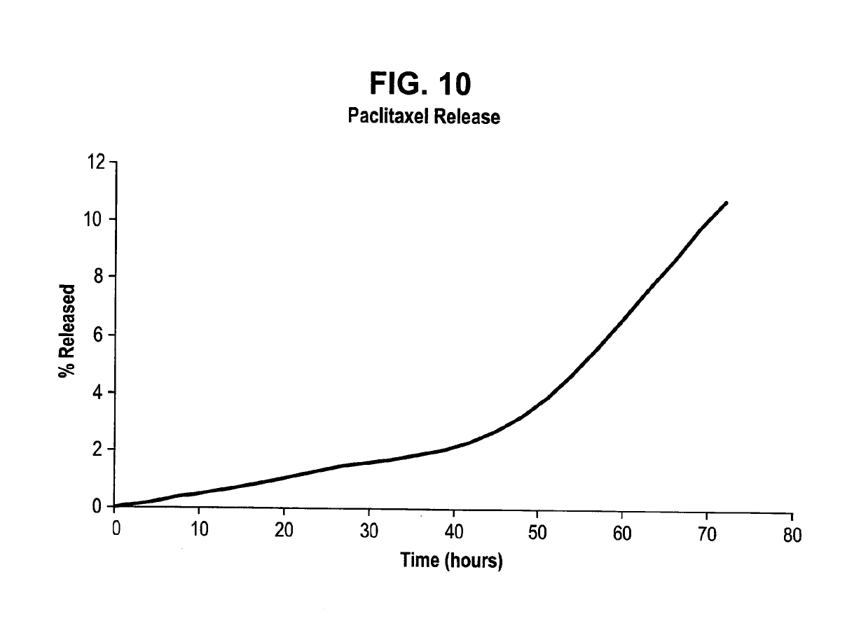

The release profile of representative nanoparticles of the present invention was determined. Nanoparticles prepared according to example 2 with paclitaxel content of 3 wt % in a solution (2 ml) of 0.1M phosphate buffered saline (PBS) and 10% ethanol was introduced into a dialysis bag (8-10 kDa). The dialysis bag was submerged in 4 ml of 0.1M PBS at 37° C. with stirring at 150 rpm. The percentage of paclitaxel released was measured at a series of time points. Results are shown in Table 11 and FIG. 10.

TABLE 11

| Time elapsed (h) | Paclitaxel released (%) |
|---|---|
| 0 | 0 |
| 6 | 0.312 |
| 24 | 1.32 |
| 48 | 3.24 |
| 72 | 10.828 |

1. A nanoparticle comprising a block copolymer
comprising blocks A and D, wherein block A comprises a first polymer comprising monomer units B and C, wherein B is an aliphatic dicarboxylic acid wherein the total number of carbon atoms is ≦30 and C is a dihydroxy or diamino monomer,
and wherein block D comprises a second polymer comprising a hydrocarbon chain containing ester or ether bonds with hydroxyl number ≧10.

2. A nanoparticle according to claim 1, wherein A has the formula —[(B—C)ₙ—B]— wherein n is a numerical index of at least 1, selected independently for each block A.

3. A nanoparticle according to claim 1, wherein C is a straight-chain aliphatic diol comprising from 4 to 10 carbon atoms.

4. A nanoparticle according to claim 1, wherein C is 1,8-octanediol.

5. A nanoparticle according to claim 1, wherein B comprises from 4 to 10 carbon atoms.

6. A nanoparticle according to claim 5, wherein B comprises from 5 to 10 carbon atoms.

7. A nanoparticle according to claim 1, wherein the polymer D is selected from the group consisting of polyethylene glycols, polyamidoamines, polyamines, polyols and combinations thereof.

8. A nanoparticle according to claim 1, wherein the nanoparticle has incorporated at least one active agent.

9. A nanoparticle according to claim 8, wherein the at least one active agent has a log P value of −1.0 to +5.6.

10. A nanoparticle according to claim 8, wherein the at least one active agent is selected from the group comprising docetaxel and paclitaxel.

11. A nanoparticle according to claim 1, wherein the nanoparticle comprises one or more coupling agent(s) suitable for covalently attaching one or more surface-modifying agent(s) to said nanoparticle.

12. A nanoparticle according to claim 11, wherein the nanoparticle has associated or incorporated at least one surface-modifying agent.

13. A nanoparticle according to claim 12, wherein said at least one surface-modifying agent is selected from the group consisting of a diagnostic agent, a targeting agent, an imaging agent, and a therapeutic agent.

14. A nanoparticle according to claim 12, wherein the at least one surface-modifying agent is selected from the group comprising thiolated polymers, fluorophors, BBB signal peptides and RGDS.

15. A nanoparticle according to claim 12, wherein the at least one surface-modifying agent is a peptide comprising one of SEQ ID NOS:1-4.

16. A nanoparticle according to claim 12, wherein the surface-modifying agent is covalently attached via a coupling agent selected from the group consisting of: 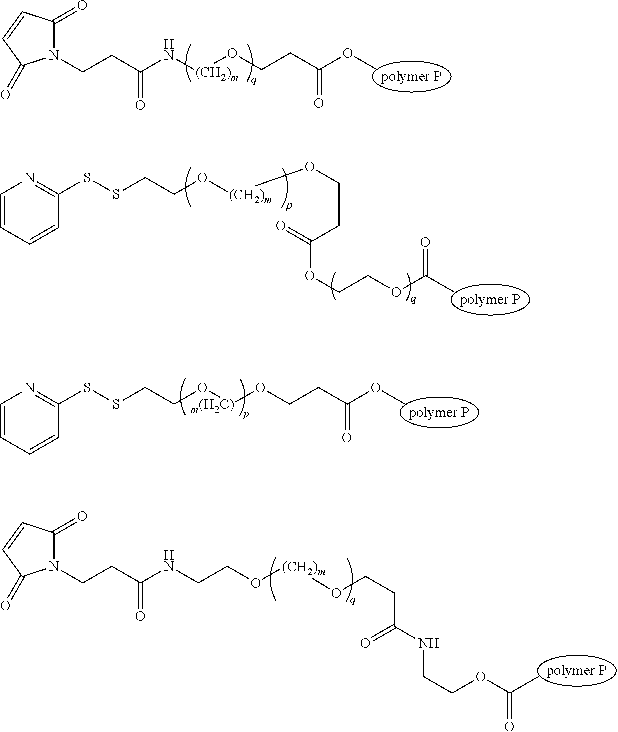
wherein m is a numerical index equal to or greater than 1; p is a numerical index greater than 1; q is a numerical index greater than 1; and “polymer P” is the block copolymer.

17. A nanoparticle according to claim 11, wherein the coupling agent is a group of Formula (IV):

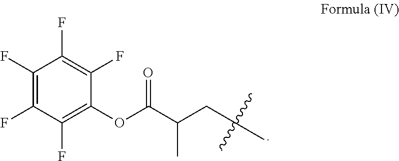 [SMILES: *CC(C)C(=O)Oc1c(F)c(F)c(F)c(F)c1F]

18. A composition comprising a nanoparticle of claim 1 and a vehicle.

19. A composition according to claim 18, which is a pharmaceutical composition wherein said vehicle is a pharmaceutically acceptable diluent or excipient.

20. A composition according to claim 18, wherein the vehicle is a polar liquid.

21. A composition claim 18, wherein the vehicle is a biological fluid.

22. A method for preparing the nanoparticle of claim 1, comprising:
i) dissolving the block copolymer in a diffusing medium to form a first solution;
ii) mixing said first solution with a dispersing medium to form precipitated nanoparticles comprising said block copolymer, and a liquid phase comprising the diffusing and dispersing media; and
iii) separation of the nanoparticles from the liquid phase, wherein the diffusing medium comprises a solvent in which the block copolymer is soluble, wherein the dispersing medium comprises a solvent in which the block copolymer is not soluble, and wherein the diffusing medium and the dispersing medium are miscible.

23. A method for preparing the composition of claim 18, comprising:
i) dissolving the block copolymer in a diffusing medium to form a first solution;
ii) mixing said first solution with a dispersing medium to form precipitated nanoparticles comprising said block copolymer and a liquid phase comprising the diffusing and dispersing media;
iii) separating the nanoparticles from the liquid phase, wherein the diffusing medium comprises a solvent in which the block copolymer is soluble, wherein the dispersing medium comprises a solvent in which the block copolymer is not soluble, and wherein the diffusing medium and the dispersing medium are miscible; and
iv) re-suspending the nanoparticles in a vehicle.

24. A method for preparing the nanoparticle of claim 8, wherein the method comprises use of at least one liquid medium comprising the active agent(s) dissolved therein.

25. A method for preparing the nanoparticle of claim 8, said method comprising the steps of:
i) producing nanoparticles;
ii) incubating said nanoparticles with a concentrated solution of the active agent(s); and
iii) separating the nanoparticles comprising said active agent(s) from the liquid phase.

26. The method of claim 25, further comprising the step of:
iv) re-suspending the nanoparticles in a vehicle.
